SEQUENCE LISTING (5 sequences)

SEQ ID NO: 1 - DNA, 61 bp, Artificial Sequence
  Description of Artificial Sequence Synthetic oligonucleotide
ttatcagtcgacggtaccggacatatgcccgggaattcggccattacggcctgcaggatcc

SEQ ID NO: 2 - DNA, 74 bp, Artificial Sequence
  Description of Artificial Sequence Synthetic oligonucleotide
ggatccggccgcctcggccctcgagaagctttctagaccattcgtttggcgcgcgggcccagtaggtaagtgaa

SEQ ID NO: 3 - DNA, 21 bp, Artificial Sequence
  Description of Artificial Sequence Synthetic oligonucleotide
ggatgcagaaggagatcactg

SEQ ID NO: 4 - DNA, 20 bp, Artificial Sequence
  Description of Artificial Sequence Synthetic oligonucleotide
cgatccacacggagtacttg

SEQ ID NO: 5 - DNA, 20 bp, Artificial Sequence
  Description of Artificial Sequence Synthetic oligonucleotide
ccctggcacccagcacaatg

(19) United States
(12) Patent Application Publication
(10) Pub. No.: US 2012/0149785 A1
(43) Pub. Date: Jun. 14, 2012

(54) METHOD OF ESTIMATING SEPSIS RISK IN AN INDIVIDUAL WITH INFECTION

(75) Inventors: Thomas Ryan, Castleknock (IE); Mary White, Salthill Co. (IE); Owen Ross McManus, Killiney (IE); Dermot Kelleher, Dun Laoghaire (IE)

(73) Assignee: The Provost, Fellows and Scholars of the College of the Holy and Undivided Trinity of Queen Elizabe, Dublin (IE)

(21) Appl. No.: 13/123,559

(22) PCT Filed: Oct. 9, 2009
(86) PCT No.: PCT/IE2009/000070

§ 371 Date: Oct. 21, 2011

(30) Foreign Application Priority Data
Oct. 9, 2008 (IE) 2008/0820

(51) Int. Cl. C12Q 1/68; A61K 35/00; A61P 31/00; G01N 21/59

(52) U.S. Cl. 514/789; 435/6.17; 435/6.12

(57) ABSTRACT

A method of estimating sepsis risk in an individual with infection comprises a step of assaying a biological sample from the individual for an IL-2 or IL-7 mRNA value, and correlating the mRNA value with sepsis risk. The IL-2 and IL-7 mRNA values are quantified by absolute quantification of mRNA copy number, wherein the copy numbers are normalised to a house keeping gene and corrected against a calibration curve for serial dilutions of the IL-2 and IL-7 cDNA. The method generally involves a step of assaying a biological sample from the individual for IL-2 and/or IL-7 mRNA values, optionally in combination with mRNA values for other cytokines, and correlating a sum or difference of the values with sepsis risk using a regression analysis curve against outcome.

### INTRODUCTION

The invention relates to a method of estimating the risk of an individual with infection developing severe sepsis. The invention also relates to a method of discriminating between a patient with infection who is unlikely to develop severe sepsis, and a patient with infection who is likely to develop severe sepsis.

### STATEMENT OF INVENTION

According to the invention, there is provided a method of estimating sepsis risk in an individual comprising a step of assaying a biological sample from the individual for an IL-2 or IL-7 mRNA value, and correlating the mRNA value with sepsis risk.

As used herein, the term “sepsis risk” should be understood to mean the risk that an individual will develop sepsis. Generally, the individual will have an infection, in which case the term should be taken to mean the risk that the individual will develop sepsis in response to infection. In other cases, the method may be employed to monitor an at-risk patient to determine whether their risk of developing sepsis is changing. This may be carried out, for example, in the situation where an individual has received an at-risk prognosis, and is being treated to avoid development of sepsis, wherein the method of the invention is employed to monitor sepsis risk.

In this specification, the term “infection” should be taken to include any disease or illness which is induced or caused by the presence of organisms in tissue, bodily fluid or cavity. In this specification, the term “sepsis” or “severe sepsis” are used interchangeably, and should be taken to mean the occurrence of an overwhelming illness with failure of bodily organ systems, which may be remote from the site of infection. These failing organ systems include but are not limited to the respiratory system, the cardiovascular system, the renal system, the hepatic, coagulation systems and the central nervous system. This definition is in accordance with consensus definition of severe sepsis and sepsis (American College of Chest Physicians/Society of Critical Care Medicine Consensus Conference: definitions for sepsis and organ failure and guidelines for the use of innovative therapies in sepsis. Crit Care Med, 1992. 20(6): p. 864-74.)

The term “biological sample” may be any sample obtained from an individual such as, for example, blood, serum, saliva, urine, cerebrospinal fluid, tissue, cells, etc. In a preferred embodiment of the invention, the sample will be a lymphocyte preparation such as lymphocytes from a peripheral blood sample, especially lymphocytes derived from the buffy coat layer of a peripheral blood sample (which is rich in T-cells and monocytes). In many cases, the individual will be a person with an established infection, or a person at risk of developing an infection, such as a patient who is immunocompromised due to disease, surgery or other factors. In other cases, the individual may be a person known to have an infection, or severe sepsis, and who is under going a therapeutic treatment regime, in which case the method of the invention may be employed to monitor the effectiveness of the treatment. In most cases, the individual will be human, however the use of the invention with higher mammals is not excluded.

Suitably, the IL-2 and IL-7 mRNA value is quantified by absolute quantification of mRNA copy number (or a function of copy number), wherein the copy numbers are normalised to a house keeping gene. PCR is generally employed, especially quantitative PCR (i.e. real time PCR), in which the PCR process is typically calibrated against serial dilutions of a known quantity of the cDNA of the respective cytokine. The mRNA value may be a normalised mRNA copy number, or a function of a normalised mRNA copy number, for example the Log 10 of the normalised mRNA copy number. The housekeeper gene employed for normalisation is selected generally selected from β-actin and GAPDH, although other housekeeping genes will be known to the person skilled in the art. Generally, the values for mRNA are normalised against a housekeeping gene and corrected against a calibration curve for serial dilutions of the respective cytokine cDNA.

The present application is based on the surprising finding that IL-2 and IL-7 mRNA levels vary between individuals with infection, individuals with sepsis, and healthy individuals and, as such, may be used as prognostic biomarkers to predict the risk of development of sepsis. Thus, IL-2 and/or IL-7 levels may be used as prognostic variables of sepsis, optionally in combination with other cytokine mRNA levels. Cytokine levels may be represented in a number of different ways, for example mRNA copy number, or a function of the mRNA copy number, for example, Log 10 of the mRNA copy number, and the mRNA copy number for a given cytokine will vary depending on the housekeeping gene employed to normalise the copy number. Moreover, the present invention encompasses a number of different ways of correlating the mRNA values with sepsis risk, including: correlating IL-2 or IL-7 absolute mRNA copy number with risk of sepsis; correlating the sum of IL-2 and IL-7 absolute mRNA copy number (or a function of copy number) with risk of sepsis; correlating a sum of at least two pro-inflammatory cytokine mRNA values (including at least one of IL-2 and IL-7, and preferably at least one of IL-23 and Interferon-γ) with risk of sepsis; and correlating the difference between (a) a mRNA value of at least one pro-inflammatory cytokine (including at least one of IL-2 and IL-7) and (b) a mRNA value of at least one anti-inflammatory cytokine, with risk of sepsis. A number of different algorithms are provided herein for correlating IL-2 and/or IL-7 mRNA values, optionally in combination with mRNA values for other cytokines, with risk of sepsis.

In one embodiment, the method involves a step of assaying a biological sample from the individual for IL-2 and IL-7 mRNA values, and correlating a sum of the values with sepsis risk. Typically, the mRNA values are provided in the form of a function of the (normalised) mRNA copy number, for example the Log 10 of the normalised copy number. Thus, the IL-2 mRNA value is represented by the Log 10 of the IL-2 mRNA copy number, and the IL-7 mRNA value is represented by the Log 10 of the IL-7 mRNA copy number, wherein the sum of the mRNA values is correlated with a numerical scale, for example 3 to 8.5, to provide sepsis risk, wherein 8.5 typically represents low sepsis risk and 3.5 typically represents high sepsis risk.

In another embodiment, the method involves a step of assaying a biological sample from the individual for a mRNA value of at least two pro-inflammatory cytokines including at least one of IL-2 and IL-7, and optionally at least one of IL-23 and Interferon-γ (INF), and correlating a sum of the mRNA values with sepsis risk. Typically, the step of correlating the sum of mRNA values with sepsis risk comprises the step of correlating the sum using a logistic regression analysis curve against outcome. Suitably, the mRNA value is a normalised mRNA copy number or a function of the normalised mRNA copy number, for example, the Log 10 of the mRNA copy number.

Preferably, the at least two pro-inflammatory cytokines are selected from the group consisting of: IL-2 and IL-23; IL-2 and INF; IL-7 and IL-23; IL-7 and INF; IL-2, IL-7 and IL-23; IL-2, IL-7 and INF; IL-2, IL-23, and INF; and IL-7, IL-23, and INF.

Thus, for example, the at least two pro-inflammatory cytokines may be IL-2 and IL-23, and wherein the sum of the Log 10 of the mRNA values is correlated with a numerical scale, suitably of 5 to 9 or 4 to 8, to provide sepsis risk, in which 9 typically represents low sepsis risk and 5 typically represents high sepsis risk.

Alternatively, the at least two pro-inflammatory cytokines may be IL-2 and INF, and wherein the sum of the Log 10 of the mRNA values is correlated with a numerical scale, suitably of 2.5 to 8 to 3.5 to 7, to provide sepsis risk, in which 8 typically represents low sepsis risk and 2.5 typically represents high sepsis risk.

Alternatively, the at least two pro-inflammatory cytokines may be IL-7 and IL-23, and wherein the sum of the Log 10 of the mRNA values is correlated with a numerical scale, suitably of 6 to 10.5 or 7 to 10, to provide sepsis risk, wherein 10.5 typically represents low sepsis risk and 6 typically represents high sepsis risk.

Alternatively, the at least two pro-inflammatory cytokines may be IL-7 and INF, and wherein the sum of the Log 10 of the mRNA values is correlated with a numerical scale, typically of 3.5 to 8.5, to provide sepsis risk, wherein 8.5 typically represents low sepsis risk and 3.5 typically represents high sepsis risk.

In a further embodiment, the method of the invention involves a step of assaying a biological sample from the individual for a mRNA value of at least one pro-inflammatory cytokine, including at least one of IL-2 and IL-7, and at least one anti-inflammatory cytokine, preferably (but not necessarily) selected from IL-10 and IL-27, calculating the difference between the pro-inflammatory mRNA value and the anti-inflammatory mRNA value, and correlating the difference with sepsis risk. The difference is the mRNA value of the pro-inflammatory cytokine minus the mRNA value of the ant-inflammatory cytokine. Where there is more than one pro-inflammatory cytokine, a composite mRNA value is provided (i.e. mRNA value of one cytokine plus the mRNA value of the other cytokine(s). Likewise, where there is more than one anti-inflammatory cytokine, a composite mRNA value is provided (i.e. mRNA value of one cytokine plus the mRNA value of the other cytokine(s).

Thus, two or more pro-inflammatory cytokines are typically employed, wherein the mRNA value for each of the two or more pro-inflammatory cytokines are summated to provide a composite pro-inflammatory mRNA value. Likewise, where two or more anti-inflammatory cytokines are employed, the mRNA values for each of the two or more anti-inflammatory cytokines are summated to provide a composite anti-inflammatory mRNA value.

Preferably, the two more pro-inflammatory cytokines includes at least one of IL-2 or IL-7, and one or more of IL-23 and INF.

Suitably, the step of correlating the sum of mRNA values with sepsis risk comprises the step of correlating the sum using a logistic regression analysis curve against outcome. Typically, the mRNA value is a normalised mRNA copy number or a function of the normalised mRNA copy number, such as a Log 10 of the mRNA copy number.

The pro-inflammatory and anti-inflammatory combination is suitably selected from the group consisting of: IL-2 and IL-10; IL-2 and IL-27; IL-2, IL-23, INF, and IL-10; IL-2, IL-23, INF, and IL-27; IL-7 and IL-10; IL-7 and IL-27; IL-7, IL-23, INF, and IL-10; IL-7, IL-23, INF, and IL-27.

Thus, for example, the pro-inflammatory cytokine may comprises IL-2 and the anti-inflammatory cytokine may comprise IL-10, and wherein the difference of the Log 10 of the pro-inflammatory and anti-inflammatory mRNA values is correlated with a numerical numerical scale, typically of −3.5 to 1.5 or −2.5 to 1, to provide sepsis risk, in which 1.5 typically represents low sepsis risk and −3.5 typically represents high sepsis risk. Alternatively, the pro-inflammatory cytokine may comprise IL-7 and the anti-inflammatory cytokine may comprise IL-10, and wherein the difference of the Log 10 of the pro-inflammatory and anti-inflammatory mRNA values is correlated with a numerical scale, typically of −1 to 2.5 or 0 to 1.5, to provide sepsis risk, in which 2.5 typically represents low sepsis risk and −1 typically represents high sepsis risk.

Alternatively, the pro-inflammatory cytokines may comprise IL-2, IL-23, and INF, and the anti-inflammatory cytokine may comprise IL-10, and wherein the difference of the Log 10 of the pro-inflammatory and anti-inflammatory mRNA values is correlated with a numerical numerical scale, typically of 3.5 to 9.5 or 4 to 8, to provide sepsis risk, in which 9.5 typically represents low sepsis risk and 3.5 typically represents high sepsis risk. For the avoidance of doubt, the pro-inflammatory mRNA value is the sum of the Log 10 of the IL-2, IL-23 and INF mRNA copy numbers, which copy numbers are typically normalised against β-actin.

Alternatively, the pro-inflammatory cytokines may comprise IL-7, IL-23, and INF, and the anti-inflammatory cytokine may comprise IL-10, and wherein the difference of the Log 10 of the pro-inflammatory and anti-inflammatory mRNA values is correlated with a numerical scale, typically of 4.5 to 10.5, to provide sepsis risk, in which 10.5 typically represents low sepsis risk and 4.5 typically represents high sepsis risk. For the avoidance of doubt, the pro-inflammatory mRNA value is the sum of the Log 10 of the IL-2, IL-23 and INF mRNA copy numbers.

Alternatively, the pro-inflammatory cytokines may comprise IL-7, IL-23, and INF, and the anti-inflammatory cytokines comprise IL-10 and Il-27, and wherein the difference of the Log 10 of the pro-inflammatory and anti-inflammatory mRNA values is correlated with a numerical scale, typically of 1.5 to 7, to provide sepsis risk, in which 7 typically represents low sepsis risk and 1.5 typically represents high sepsis risk. For the avoidance of doubt, the pro-inflammatory mRNA value is the sum of the Log 10 of the IL-2, IL-23 and INF mRNA copy numbers, and the anti-inflammatory mRNA value is the sum of the Log 10 of the IL-7 and IL-10 mRNA copy numbers.

In a preferred embodiment, the invention relates to a method of discrimination between a patient with infection who is unlikely to develop severe sepsis and a patient with infection who is likely to develop severe sepsis, the method comprising a step of assaying a biological sample from the individual for an expression level of IL-2 mRNA and/or IL-7 mRNA, wherein the level of expression of IL-2 mRNA and/or IL-7 mRNA correlates with a likelihood of the patient developing severe sepsis. Thus, for example, when a high level of IL-2 or IL-7 mRNA expression is identified, this correlates with a likelihood of the patient not developing severe sepsis. Alternatively, where a low level of IL-2 or IL-7 mRNA expression is determined, this correlates with likelihood that the patient will develop severe sepsis. Assessing the risk that a patient may develop severe sepsis is important as it will help a clinician decide whether the patient needs to be treated in an ICU, and whether aggressive antibiotic or other therapies are required.

In one embodiment of the invention, the method of the invention involves a step of assaying a biological sample from the individual for an expression level of IL-2 and IL-7 mRNA, and correlating the determined levels with a likelihood of the patient developing/not developing severe sepsis.

The levels of IL-2 and IL-7 mRNA present in biological samples, especially in patients with infection and severe sepsis, tend to be very low. Accordingly, the method employed in assaying a biological sample for the levels of these cytokines is required to be extremely sensitive. Cytokine mRNA values are quantified by absolute quantification of mRNA copy number, wherein the copy numbers are ideally normalised to a house keeping gene such as, for example, GAPDH or b-Actin, and corrected against a calibration curve for serial dilutions of the respective cytokine cDNA. Other suitable housekeeper genes will be known to those skilled in the art. Using this method, and β-actin as the housekeeping gene, a high level of IL-2 mRNA expression correlates with IL-2 copy number of at least 570 copy numbers of mRNA, and a low level of IL-2 expression correlates with an IL-2 copy number of less than 172 copy numbers of mRNA. Likewise, a high level of IL-7 expression correlates with IL-7 copy number of at least 1675 copy numbers of mRNA, and a low level of IL-2 expression correlates with an IL-7 copy number of less than 283 copy numbers of mRNA.

In a preferred embodiment of the invention, the methods of the invention comprise a scoring system in which a patient is assigned a score of 1, 2 or 3 depending on whether the level of expression of IL-2 or IL-7 is high, medium or low. In the case of IL-2, a score of 1 correlates with a mRNA copy number of at least 570, a score of 2 correlates with a mRNA copy number of from 570 and 172, and a score of 3 correlates with a mRNA copy number of less than 172. A score of 3 correlates with a likelihood of developing severe sepsis, and a score of 1 correlates with a likelihood of not developing severe sepsis.

In an analysis of the relation between IL-2 derived risk category, patients in group 3 with low IL-2 mRNA copy numbers were 3.25 times more likely to have severe sepsis rather than infection when compared with patients in group 1 and 2.

In the case of IL-7, a score of 1 correlates with a mRNA copy number of at least 1675, a score of 2 correlates with a mRNA copy number of from 1675 and 283, and a score of 3 correlates with a mRNA copy number of less than 283. A score of 3 correlates with a likelihood of developing severe sepsis, and a score of 1 correlates with a likelihood of not developing severe sepsis.

In an analysis of the relation between IL-7 derived risk category, patients in groups 2 and 3 were 3.5 times more likely to have severe sepsis rather than infection when compared with patients in group 1.

Preferably, the scoring system involves assaying a patient for IL-2 and IL-7 mRNA levels, assigning a score of 1, 2 or 3 to the patient in respect of each of IL-2 and IL-7, and summating the score to provide a composite score for the patient of between 2 and 6. Typically, a score of 4, 5 or 6 indicates a likelihood that the patient has, or will develop, severe sepsis. Suitably, a score of 5 or 6 indicates a strong likelihood that the patient has, or will develop, severe sepsis. Typically, a score of 2 or 3 indicates a likelihood that the patient will not develop severe sepsis. Suitably, a score of 2 indicates a strong likelihood that the patient will not develop severe sepsis.

With respect to the probability of developing severe sepsis, these scoring systems can be used to determine relative risk, or an odds ratio. Thus when patients with the combined IL-2 and IL-7 scores of 2 and 3 are considered as a low risk group, with patients with a score of 4 as an intermediate risk group, and patients with a score of 5 or 6 as a high risk group, there is an obvious relation between risk group and response to infection. In this analysis of patients with infection and patients with severe sepsis, intermediate risk patients were 8 times more likely to have severe sepsis than low risk patients, high risk patients were 13.7 times more likely to develop severe sepsis than intermediate risk patients, and high risk patients were 110 times more likely to develop severe sepsis than low risk patients.

It will be appreciated that the values assigned above are informed by the choice of housekeeping gene against which the copy numbers are corrected, and that therefore a different housekeeping gene would likely result in a different set of values for determining “high” and “low” expression levels. It will be appreciated therefore that alternative methods of determining absolute quantification of mRNA copy numbers that employ different housekeeping genes likewise forms part of the invention.

As mentioned above, the methods of the invention may be employed to estimate risk of an individual with infection developing severe sepsis, and to stratify patients with infection into those that are unlikely to develop severe sepsis and those that are likely to develop severe sepsis. As such, the invention also relates to a method of treating or preventing severe sepsis in a patient comprising a step of determining whether the patient is likely to develop severe sepsis in response to infection according to the methods of the invention, and where it is determined that the patient is likely to develop severe sepsis, then treating the patient to treat or prevent severe sepsis.

The invention also relates to a method of monitoring the efficacy of a therapeutic or prophylactic treatment of severe sepsis, the method comprising a step of assaying a biological sample from the individual for an expression level of IL-2 mRNA and/or IL-7 mRNA, and correlating the level of expression of IL-2 mRNA and/or IL-7 mRNA with sepsis risk. Thus, for example, when the levels of the mRNA for each cytokine increase (i.e. from low to high, or from low to medium), this would be indicative of the treatment having a positive effect. Likewise, if initially high levels of the mRNA for each cytokine decrease during a course of treatment, then the likelihood would be that the treatment is not working.

### BRIEF DESCRIPTION OF THE FIGURES

FIG. 1 shows a restriction map and multiple cloning sites for pDNR-LIB vector

FIG. 2 is a map of the pDNR-LIB vector MCS, multiple cloning site. The IL2 cDNA insert replaces the stuffer fragment. Unique restriction sites are shown in bold or in colour.

FIG. 3 shows a restriction map and multiple cloning sites for pCMV-SPORT6 vector

FIG. 4 shows a DNA gel Single Digest gel with Ecor I and Xba I for IL2 and IL7 respectively—1% agarose DNA Gel—Lane 1 contains a 1 kb ladder. Lane 2 contains linear plasmid IL2 DNA following single restriction enzyme digestion with EcorI. Lane 3 contains linear plasmid IL7 DNA following single restriction enzyme with XbaI.

FIG. 5 shows a DNA gel Double Digest gel with Ecor I and HINDIII for IL2 and EcorI and XbaI IL7 respectively—1% agarose DNA Gel—Lane 1 contains a 1 kb ladder. Lane 2 contains linear plasmid IL2 DNA following double restriction enzyme digestion with EcorI and HINDIII. Lane 3 contains linear plasmid IL7 DNA following double restriction enzyme digestion with XbaI and EcorI.

FIG. 6 shows an absorbance Spectrum of plasmid DNA

FIG. 7 shows a Standard Curve for IL2.

FIG. 8 shows a Standard Curve for IL7.

FIG. 9 shows a Standard Curve for the house-keeping gene β-Actin.

FIG. 10: logistic Fit of Response to infection By Combined IL-2 and IL-7 mRNA Log base 10

FIG. 11A analysis of Summated Score A By Groups (IL-7, IL-23, INF and IL-10)

FIG. 11B: logistic Fit of Response to Infection By Summated Score A, with 0 representing sepsis and 1 representing infection.

FIG. 12A analysis of Summated Score B By Groups (IL-2, IL-23, INF and IL-10)

FIG. 12B logistic Fit of Response to Infection By Summated Score B, with 0 representing sepsis and 1 representing infection.

FIG. 13A analysis of Summated Score C By Groups (IL-7, IL-23, INF, IL-10 and IL-27)

FIG. 13B logistic Fit of Response to Infection By Summated Score C, with 0 representing sepsis and 1 representing infection.

FIG. 13C ROC Curve for Patient response to infection by Summated Score C. ROC value=0.88.

FIG. 14A analysis of Summated Score E By Groups (IL-2 and IL-10)

FIG. 14B logistic Fit of Response to Infection By Summated Score E, with 0 representing sepsis and 1 representing infection.

FIG. 15A analysis of Summated Score F By Groups (IL-2 and INF)

FIG. 15B logistic Fit of Response to Infection By Summated Score F, with 0 representing sepsis and 1 representing infection

FIG. 16A analysis of Summated Score G By Groups (IL-2 and IL-23)

FIG. 16B logistic Fit of Response to Infection By Summated Score G, with 0 representing sepsis and 1 representing infection

FIG. 17A analysis of Summated Score H By Groups (IL-7 and IL-10)

FIG. 17B logistic Fit of Response to Infection By Summated Score H, with 0 representing sepsis and 1 representing infection

FIG. 18A analysis of Summated Score I By Groups (IL-7 and INF)

FIG. 18B logistic Fit of Response to Infection By Summated Score I, with 0 representing sepsis and 1 representing infection.

FIG. 19A analysis of Summated Score J By Groups (IL-7 and IL-23)

FIG. 19B logistic Fit of Response to Infection By Summated Score J, with 0 representing sepsis and 1 representing infection.

FIG. 20 is a graph showing the IL-2 mRNA copy numbers at day 1 for the three patients groups.

FIG. 21 is a graph showing the IL-7 mRNA copy numbers at day 1 for the three patients groups.

FIG. 22 is a graph showing the IL-2 mRNA copy numbers at day 1 for the infection and severe sepsis patients groups.

FIG. 23 is a graph showing the IL-7 mRNA copy numbers at day 1 for the infection and severe sepsis patients groups.

FIG. 24 is a graph showing the IL-2 mRNA copy numbers at day 1 for the control and infection patient groups.

FIG. 25 is a graph illustrating the scoring system of the invention in which patients are scored as 1, 2 or 3 according to their expression levels of IL-2, and the scores are correlated with the severe sepsis and infection patient groups.

FIG. 26 is a graph illustrating the scoring system of the invention in which patients are scored as 1, 2 or 3 according to their expression levels of IL-2, and the scores are correlated with the control, severe sepsis and infection patient groups.

FIG. 27 is a graph illustrating the scoring system of the invention in which patients are scored as 1, 2 or 3 according to their expression levels of IL-7, and the scores are correlated with the control, severe sepsis and infection patient groups.

FIG. 28 is a graph illustrating the scoring system of the invention in which patients are scored as 1, 2 or 3 according to their expression levels of IL-7, and the scores are correlated with the severe sepsis and infection patient groups.

FIG. 29 is a graph illustrating the scoring system of the invention in which the scores for IL-2 and IL-7 are summated to provide a composite score of from 2 to 6 for the patient, and the scores are correlated with the control, infection and severe sepsis groups.

FIG. 30 is a graph illustrating the scoring system of the invention in which the scores for IL-2 and IL-7 are summated to provide a composite score of from 2 to 6 for the patient, and the scores are correlated with the infection and severe sepsis groups.

### DETAILED DESCRIPTION OF THE INVENTION

### 1. Characterisation of Patients Groups

The data contained herein is based on two distinct cohort of patients, each cohort comprising three patient groups, namely a control group of healthy volunteers, a group of patients with bacteraemia who did not develop severe sepsis and a group of patient with obvious infection who developed severe sepsis. The Bacteraemic patients that were recruited had been hospitalised with infection, and then grew gram negative organisms in blood cultures, but did not have any evidence of organ failure. The septic group of patients had obvious infection, such as pneumonia, peritonitis or cellulitis, and developed an overwhelming illness in response to infection, with the majority developing septic shock and multiple organ failure, and requiring admission to intensive care for support of failing organ systems.

Patients with obvious cause of immune compromise, such as HIV/AIDS, neutropoenia, and or high steroid dosage, were excluded from this study.

### 2. Description of Methods of Determining Copy Number

The method described below relate to determining mRNA copy number (or a function of copy number) for IL-2 and IL-7. The methods may likewise be applied for the determination of copy numbers for IL-10, IL-23, IL-27, Interferon-γ, and other cytokine (WO2007/060647)

The DNA standards for quantitative real-time per may be prepared by either cloning a PCR product that encompasses the quantified amplicon. This can be prepared by PCR from a cDNA population containing the target mRNA. Alternatively, as described below, DNA standards may be prepared by culturing E. Coli with the enclosed vector containing the relevant gene sequence, in this case IL-2 and IL-7. The ensuing DNA harvested from the plasmid is purified, IL-2 and IL-7 sequence verification and quantified, and finally the volume of plasmid DNA corresponding to copy numbers of target nucleic acid sequences is determined.

### 2.1.1 Preparation of the IL2 Standard

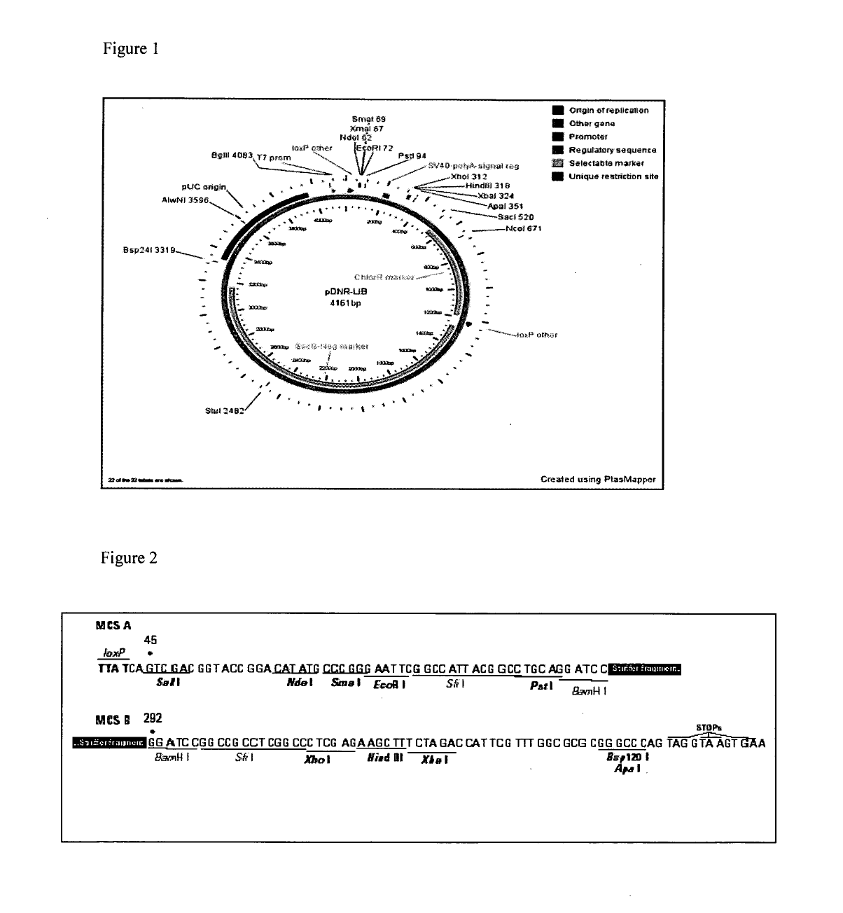

IL2 plasmid was purchased from Open Biosystems (MHS1011-98053730 Human MGC Verified FL cDNA IRAU). It consisted of an 894 bp cDNA clone inserted into a 4.161 kb pDNR-LIB vector. This is illustrated in FIGS. 1 and 2.

### 2.1.2 Preparation of the IL7 Standard

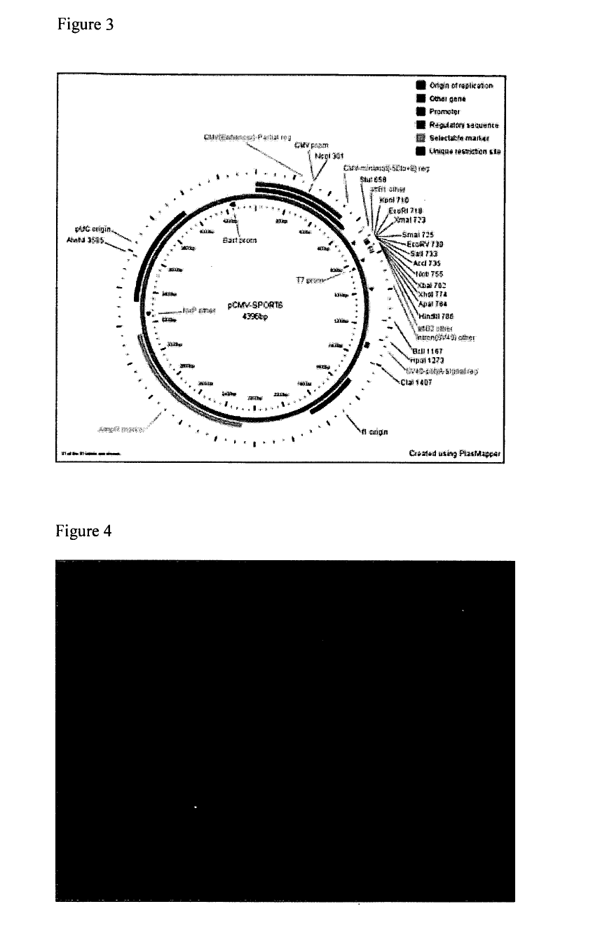

IL7 plasmid was purchased from Open Biosystems (MHS1010-9205095 Human MGC Verified FL cDNA IRAT). It consisted of a 2125 bp cDNA clone inserted into a 4396 bpDNR-LIB vector. This is illustrated in FIG. 3.

### 2.1.3 Plasmid Culture Conditions

An E. coli culture harbouring the pDNR-LIB vector containing the IL-2 gene was streaked onto a chloramphenicol (25 μg/ml) containing LB agar plate and incubated at 37° C. overnight. A similar E. coli culture harbouring the pCMV-SPORT6 vector containing the IL-7 gene was streaked onto an ampicillin (100 μg/ml). A single colony was isolated from each plate and streaked onto another plate. A well-isolated colony from this second plate was then used to inoculate a liquid L-broth culture grown overnight at 37° C. for each plasmid. These steps ensure the isolation of a clone of a single bacterium.

### 2.1.4 Purification of DNA

The Fast Ion Plasmid Midi kit Fast Ion™ (Cat No YP125/YPM10), was used to purify plasmid DNA from 100 ml overnight cultures of E. coli according to the manufacturers' instructions. Bacteria were lysed and the cleared lysate is passed through a cation-exchange column, which binds the re-natured plasmid DNA. The column with bound DNA was washed repeatedly and the DNA is eluted in a high-salt buffer. The DNA is then further purified and desalted by precipitation with isopropanol and resuspended in ddH₂O. Purified plasmid DNA was visualized on a 1% agarose gel as shown in FIG. 4.

### 2.1.5 Agarose Gel Electrophoresis

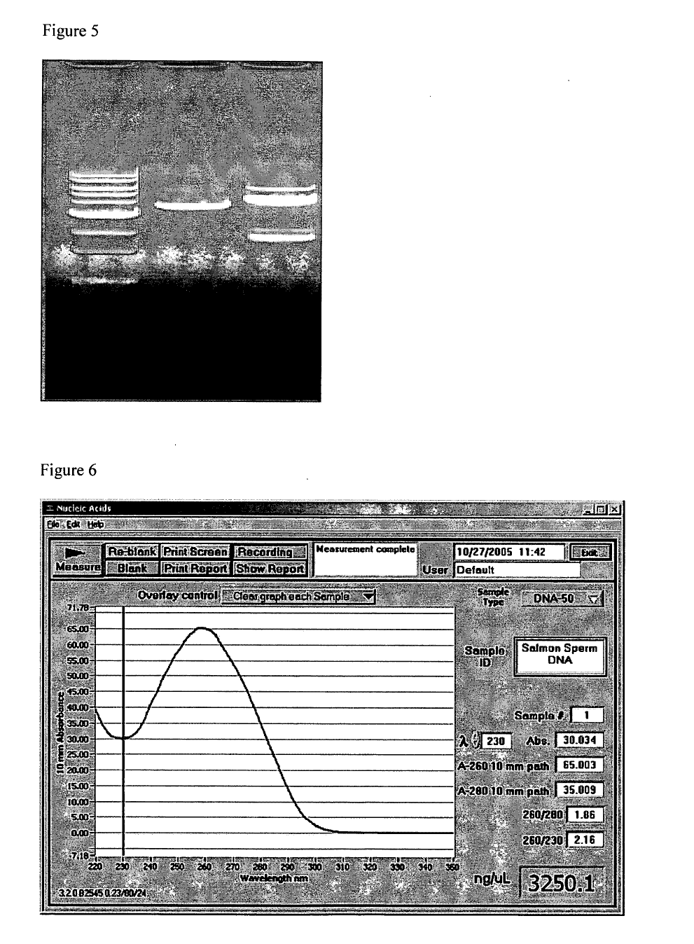

DNA samples were visualised following separation on a 1% agarose gel. Briefly, for a 1% gel, agarose (1 g) was added to 100 ml of 0.5×TBE buffer (44.5 mM tris borate, pH 8.3, 1 mM E DTA) and heated to 100° C. to dissolve the agarose. Ethidium bromide was added to a final concentration of 1 μg/ml and the molten gel was poured into a gel mould and allowed to set. DNA samples were prepared by adding an appropriate volume of 5× sample loading buffer (25 mM tris pH 7.6, 30% (v/v) glycerol, 0.125% (w/v) bromophenol blue) and these samples were electrophoresed through the gel at 135 V for 45 min in 0.5×TBE buffer. The separated DNA fragments were photographed while illuminated under UV light (FIG. 5).

### 2.1.6 Restriction Endonuclease Digestion of DNA

All restriction digests were carried out using enzymes supplied by New England Biolabs (NEB) according to the manufacturers' instructions. Briefly, 0.1-2 μg of purified DNA was incubated with 10-20 U of restriction enzyme in the appropriate NEB buffer for 2 h at the appropriate temperature. Digests with double enzymes were carried out in the recommended double digest buffer, in which all enzymes had 100% activity (FIG. 2.4).

### 2.1.7 Clone Verification

The IL2 and IL7 clone was end sequenced by MWG-Biotech, Ebersberg, Germany. This was verified against the GeneBank sequence for IL2 and IL7 using BLAST.

### 2.1.8 Quantification of DNA

DNA was quantified and qualified using the Nanodrop® ND 8000 (220-750 nm) full spectrum spectrophotometer. Briefly a 1 μl sample of DNA was placed on the measuring pedestal. The pedestal is actually the end of a fiber optic cable (receiving fibers). A second set of fiber optic cables (the source fibers) are then brought in contact with the liquid sample, causing the liquid to bridge the gaps between the fiber optic ends. A pulsed xenon flash lamp provides the light source and a spectrometer using a linear CCD array is used to analyse the light that passes through the samples. Absorbance measurements, measure any molecules absorbing at a specific wavelength. Nucleotides, RNA, ssDNA and dsDNA all absorb at 260 nm and contribute to the overall absorbance. The ratio of absorbance at 260 nm and 280 nm is used to assess the purity of DNA and RNA. A ratio of ˜1.8 is accepted as “pure” for DNA; a ratio ˜2.0 is accepted as “pure” for RNA. If the ratio is lower it indicates the presence of contaminants. An additional measure of nucleic acid incorporates absorbance at 230 nm, with the 260/230 ratio is used as a secondary measure of nucleic acid purity. The 260/230 values for “pure” nucleic acid are expected to be in the range of 2.0-2.2. The absorbance spectrum in FIG. 2.5 indicates a DNA concentration of 3250.1 ng/μl, with a 260-280 ratio of 1.86 and a 260-230 ratio of 2.16. The absorbance spectrum for IL2 and IL7 are presented in table 1 and FIG. 6.

TABLE 1
Results from absorbance spectrum

| | A260 | A280 | | | Conc |
|---|---|---|---|---|---|
| Sample | 10 mm path | 10 mm path | 260/280 | 260/230 | ng/μl |
| IL2 | 10.314 | 5.548 | 1.86 | 2.05 | 515.7 |
| IL7 | 67.852 | 37.271 | 1.83 | 2.08 | 3365.6 |

2.1.9 Determining the Volume of Plasmid DNA Corresponding to Copy Numbers of Target Nucleic Acid Sequences, i.e. Creating a Standard Curve with a Plasmid DNA Template.

To prepare a standard curve from in which both, the cloned IL2 and IL7 sequence is present in 10*8 to 10*0 copies correspondingly. This standard curve is utilised to calculate absolute copy numbers of IL2 and IL7 in patient samples. Our quantitative real time per reactions are set up such that 1.5 μl of plasmid DNA is pipetted into each QRT-PCR reaction.

### 2.1.9.1 IL2 Standard Curve

The stock of IL2 plasmid DNA was determined to be 515.7 ng/μl by spectrophotometric analysis. The vector size for pDNR-LIB is 4161 bp. The IL2 cloned insert is 814 bp. The size of the vector+insert=4975 bp.

First we calculate the mass of a single plasmid molecule. The size of the entire plasmid (plasmid+insert) is used in this calculation using DNA Mass Formula.

m=(n)(1.096×10⁻²¹ g/bp);
m=mass
n=plasmid size (bp)

In the case of IL2:

m=4975 bp (1.096×10⁻²¹) g/bp
m=5.453×10⁻¹⁸ g=mass of a single plasmid molecule.

We then calculate the mass of plasmid containing the copy numbers of interest, in this example 10⁸:

### E.g.

Copy number (CN) of interest×mass of single plasmid=mass of plasmid DNA needed for Copy Number of interest

(10*8 CN)×(5.453×10⁻¹⁸ g)=5.453×10⁻¹⁰ g

Therefore, the mass of plasmid DNA needed for 10*8 copy numbers=5.453×10⁻¹⁰ g We then calculate the concentrations of plasmid DNA needed to achieve the copy numbers of interest (table 2).

TABLE 2

| Copy Number of Interest | Mass of Plasmid DNA (g) |
|---|---|
| 10*8 | 5.453 × 10⁻¹⁰ |
| 10*7 | 5.453 × 10⁻¹¹ |
| 10*6 | 5.453 × 10⁻¹² |
| 10*5 | 5.453 × 10⁻¹³ |
| 10*4 | 5.453 × 10⁻¹⁴ |
| 10*3 | 5.453 × 10⁻¹⁵ |
| 10*2 | 5.453 × 10⁻¹⁶ |
| 10*1 | 5.453 × 10⁻¹⁷ |

We next calculate the concentrations of plasmid DNA needed to achieve the copy numbers of interest, dividing the mass needed for respective copy number of interest by the volume pipetted into each reaction (1.5 μl) see table 3.

TABLE 3

| | | | Final Conc |
|---|---|---|---|
| Copy Number | Mass of Plasmid | Volume used in | of plasmid |
| of Interest | DNA needed (g) | each QRT-PCR μl | DNA(g/μl) |
| 10*9 | 5.453 × 10⁻⁹ | 1.5 | 3.635 × 10⁻⁹ |
| 10*8 | 5.453 × 10⁻¹⁰ | 1.5 | 3.635 × 10⁻¹⁰ |
| 10*7 | 5.453 × 10⁻¹¹ | 1.5 | 3.635 × 10⁻¹¹ |
| 10*6 | 5.453 × 10⁻¹² | 1.5 | 3.635 × 10⁻¹² |
| 10*5 | 5.453 × 10⁻¹³ | 1.5 | 3.635 × 10⁻¹³ |
| 10*4 | 5.453 × 10⁻¹⁴ | 1.5 | 3.635 × 10⁻¹⁴ |
| 10*3 | 5.453 × 10⁻¹⁵ | 1.5 | 3.635 × 10⁻¹⁵ |
| 10*2 | 5.453 × 10⁻¹⁶ | 1.5 | 3.635 × 10⁻¹⁶ |
| 10*1 | 5.453 × 10⁻¹⁷ | 1.5 | 3.635 × 10⁻¹⁷ |

The final step is to prepare a serial dilution of the plasmid DNA. The cloned sequences are highly concentrated in purified plasmid DNA stocks. A series of serial dilutions are performed if necessary to achieve a working stock of plasmid DNA for quantitative RT PCR applications.

Once the plasmid is at a workable concentration, the following formula is used to calculate the volume needed to prepare the 10*8 copy standard dilution. (in case of IL2 dilution III)

C₁V₁=C₂V₂

TABLE 4

| | | | Volume of | | | | |
|---|---|---|---|---|---|---|---|
| | Source of | Initial Conc | plasmid DNA | Vol of | Final | | Resulting |
| | Plasmid DNA | (g/μl) | (μl) | Diluent | Volume | Final Conc | Copy |
| Dilution | for dilution | (C₁) | (V₁) | (μl) | (μl) (V₂) | (g/μl) (C₂) | Numbers |
| I | Stock | 51.57 × 10⁻⁹ | 10 | 990 | 1000 | 5.157 × 10⁻⁹ | N/A |
| II | Dil I | 5.157 × 10⁻⁹ | 70.486 | 29.514 | 100 | 3.635 × 10⁻⁹ | 10*⁹ |
| III | Dil II | 3.635 × 10⁻⁹ | 10 | 90 | 100 | 3.635 × 10⁻¹⁰ | 10*⁸ |
| IV | Dil III | 3.635 × 10⁻¹⁰ | 10 | 90 | 100 | 3.635 × 10⁻¹¹ | 10*⁷ |
| V | Dil IV | 3.635 × 10⁻¹¹ | 10 | 90 | 100 | 3.635 × 10⁻¹² | 10*⁶ |
| VI | Dil V | 3.635 × 10⁻¹² | 10 | 90 | 100 | 3.635 × 10⁻¹³ | 10*⁵ |
| VII | Dil VI | 3.635 × 10⁻¹³ | 10 | 90 | 100 | 3.635 × 10⁻¹⁴ | 10*⁴ |
| VIII | Dil VII | 3.635 × 10⁻¹⁴ | 10 | 90 | 100 | 3.635 × 10⁻¹⁵ | 10*³ |
| IX | Dil VIII | 3.635 × 10⁻¹⁵ | 10 | 90 | 100 | 3.635 × 10⁻¹⁶ | 10*² |
| X | Dil IX | 3.6315 × 10⁻¹⁶ | 10 | 90 | 100 | 3.635 × 10⁻¹⁷ | 10*¹ |

The dilutent used in these dilutions was sterile TE buffer (10 mM Tris HCL, 1 mM EDTA pH 8.0 with 10 μg/ml double stranded herring DNA (sigma).

Dilutions II to X were used for quantitative PCR application.

### 2.1.9.2 IL 7 Standard Curve

The stock of IL7 plasmid DNA was determined to be 3365.6 ng/μl by spectrophotometric analysis. The vector size for pCMV-SPORT6 is 4396 bp. The IL2 cloned insert is 2125 bp. The size of the vector+insert=6521 bp.

First we calculate the mass of a single plasmid molecule. The size of the entire plasmid (plasmid+insert) is used in this calculation using DNA Mass Formula.

m=(n)(1.096×10⁻²¹ g/bp);
m=mass
n=plasmid size (bp)

In the case of IL7:

m=6521 bp (1.096×10⁻²¹) g/bp
m=7.147×10¹⁸ g=mass of a single plasmid molecule.

We then calculate the mass of plasmid containing the copy numbers of interest, in this example 10⁸:

Copy number (CN) of interest×mass of single plasmid=mass of plasmid DNA needed for Copy Number of interest

(10*8 CN)×(7.147×10¹⁸ g)=7.147×10⁻¹⁰ g

Therefore, the mass of plasmid DNA needed for 10*8 copy numbers=7.147×10⁻¹⁰ g

We then calculate the concentrations of plasmid DNA needed to achieve the copy numbers of interest (table 5).

TABLE 5

| Copy Number of Interest | Mass of Plasmid DNA (g) |
|---|---|
| 10*8 | 7.147 × 10⁻¹⁰ |
| 10*7 | 7.147 × 10⁻¹¹ |
| 10*6 | 7.147 × 10⁻¹² |
| 10*5 | 7.147 × 10⁻¹³ |
| 10*4 | 7.147 × 10⁻¹⁴ |
| 10*3 | 7.147 × 10⁻¹⁵ |
| 10*2 | 7.147 × 10⁻¹⁶ |
| 10*1 | 7.147 × 10⁻¹⁷ |

We next calculate the concentrations of plasmid DNA needed to achieve the copy numbers of interest, dividing the mass needed for respective copy number of interest by the volume pipetted into each reaction (1.5 μl) see table 6.

TABLE 6

| | | | Final Conc |
|---|---|---|---|
| Copy Number | Mass of Plasmid | Volume used in | of plasmid |
| of Interest | DNA needed (g) | each QRT-PCR μl | DNA(g/μl) |
| 10*9 | 7.147 × 10⁻⁹ | 1.5 | 4.765 × 10⁻⁹ |
| 10*8 | 7.147 × 10⁻¹⁰ | 1.5 | 4.765 × 10⁻¹⁰ |
| 10*7 | 7.147 × 10⁻¹¹ | 1.5 | 4.765 × 10⁻¹¹ |
| 10*6 | 7.147 × 10⁻¹² | 1.5 | 4.765 × 10⁻¹² |
| 10*5 | 7.147 × 10⁻¹³ | 1.5 | 4.765 × 10⁻¹³ |
| 10*4 | 7.147 × 10⁻¹⁴ | 1.5 | 4.765 × 10⁻¹⁴ |
| 10*3 | 7.147 × 10⁻¹⁵ | 1.5 | 4.765 × 10⁻¹⁵ |
| 10*2 | 7.147 × 10⁻¹⁶ | 1.5 | 4.765 × 10⁻¹⁶ |
| 10*1 | 7.147 × 10⁻¹⁷ | 1.5 | 4.765 × 10⁻¹⁷ |

The final step is to prepare a serial dilution of the plasmid DNA. The cloned sequences are highly concentrated in purified plasmid DNA stocks. A series of serial dilutions are performed if necessary to achieve a working stock of plasmid DNA for quantitative RT PCR applications.

Once the plasmid is at a workable concentration, the following formula is used to calculate the volume needed to prepare the 10*8 copy standard dilution. (table 7)

C₁V₁=C₂V₂

TABLE 7

| | Source of | | Volume of | | | | |
|---|---|---|---|---|---|---|---|
| | Plasmid | Initial Conc | plasmid | Vol of | Final | Final Conc | Resulting |
| | DNA for | (μg/l) | DNA | Diluent | Volume | (μg/l) | Copy |
| Dilution | dilution | (C₁) | (μl) (V₁) | (μl) | (μl) (V₂) | (C₂) | Numbers |
| I | Stock | 33.659 × 10⁻⁷ | 14.156 | 85.844 | 100 | 4.765 × 10⁻⁷ | 10*¹¹ |
| II | Dil I | 4.765 × 10⁻⁷ | 10 | 90 | 100 | 4.765 × 10⁻⁸ | 10*¹⁰ |
| III | Dil II | 4.765 × 10⁻⁸ | 10 | 90 | 100 | 4.765 × 10⁻⁹ | 10*⁹ |
| IV | Dil III | 4.765 × 10⁻⁹ | 10 | 90 | 100 | 4.765 × 10⁻¹⁰ | 10*⁸ |
| V | Dil IV | 4.765 × 10⁻¹⁰ | 10 | 90 | 100 | 4.765 × 10⁻¹¹ | 10*⁷ |
| VI | Dil V | 4.765 × 10⁻¹¹ | 10 | 90 | 100 | 4.765 × 10⁻¹² | 10*⁶ |
| VII | Dil VI | 4.765 × 10⁻¹² | 10 | 90 | 100 | 4.765 × 10⁻¹³ | 10*⁵ |
| VIII | Dil VII | 4.765 × 10⁻¹³ | 10 | 90 | 100 | 4.765 × 10⁻¹⁴ | 10*⁴ |
| IX | Dil VIII | 4.765 × 10⁻¹⁴ | 10 | 90 | 100 | 4.765 × 10⁻¹⁵ | 10*³ |
| X | Dil IX | 4.765 × 10⁻¹⁵ | 10 | 90 | 100 | 4.765 × 10⁻¹⁶ | 10*² |
| XI | Dil X | 4.765 × 10⁻¹⁶ | 10 | 90 | 100 | 4.765 × 10⁻¹⁷ | 10*¹ |

The dilutent used in these dilutions was sterile TE buffer (10 mM Tris HCL, 1 mM EDTA pH 8.0 with 10 μg/ml double stranded herring DNA (sigma).

Dilutions IV to XI were used for quantitative PCR application.

### 2.2.0 Primers and Probes for IL2 and IL7

All primers and probes for IL2 and IL7 were synthesized at Applied Biosystems (Foster City, Calif.). Both IL2 and IL7 were obtained as a precustomised primer and probe mix. (Taqman®Gene Expression Assays ID Hs00174114_ml and for IL7 is Taqman® Gene Expression Assays ID Hs00174202_ml).

Expression of IL2 and IL7 in patient samples were normalised to 10*7 copy numbers of the house-keeping gene β-Actin. A house-keeping gene is a reference gene that acts as an internal standard or loading control. The ideal house-keeping gene should have various features: (1) The standard gene should have the same copy numbers in all cells and (2) It should be expressed in all cells. Commonly used housekeeping standards Glyceraldehyde-3-phosphate dehydrogenase mRNA, β-Actin mRNA, MHC (major histocompatibility complex I) mRNA, Cyclophilin mRNA, mRNAs for certain ribosomal proteins e.g. RPLPO (ribosomal protein, large P0), This is also known as 36B4, P0, L10E, RPPO, PRLP0, 60S acidic ribosomal protein P0, ribosomal protein L10, Arbp or acidic ribosomal phosphoprotein P0. However, the perfect housekeeping gene does not exist, therefore we used β-Actin as it has been validated for the tissue (PBMCs)—it does not change significantly in expression when PBMCs are subjected to the experimental variables used in these experiments. The β-Actin primers and probe were designed and customised as per Stordeur at al. (Stordeur et al, 2002).

The probe stock for β-Actin (40 pmol/L) was stored at −20° C. and a working dilution of 4 pmol/L, with 200 nM probe used per 20 μL QRT-PCR reaction. 300 nM of forward and reverse primers were used per 20 μL QRT-PCR reaction.

Sequences for β-Actin (J. Immune. Methods. 2002 Apr. 1; 262 (1-2):299)

| | |
|---|---|
| Forward Primer | GGATGCAGAAGGAGATCACTG |
| Reverse Primer | CGATCCACACGGAGTACTTG |
| Probe | 6Fam-CCCTGGCACCCAGCACAATG-Tamra-p |

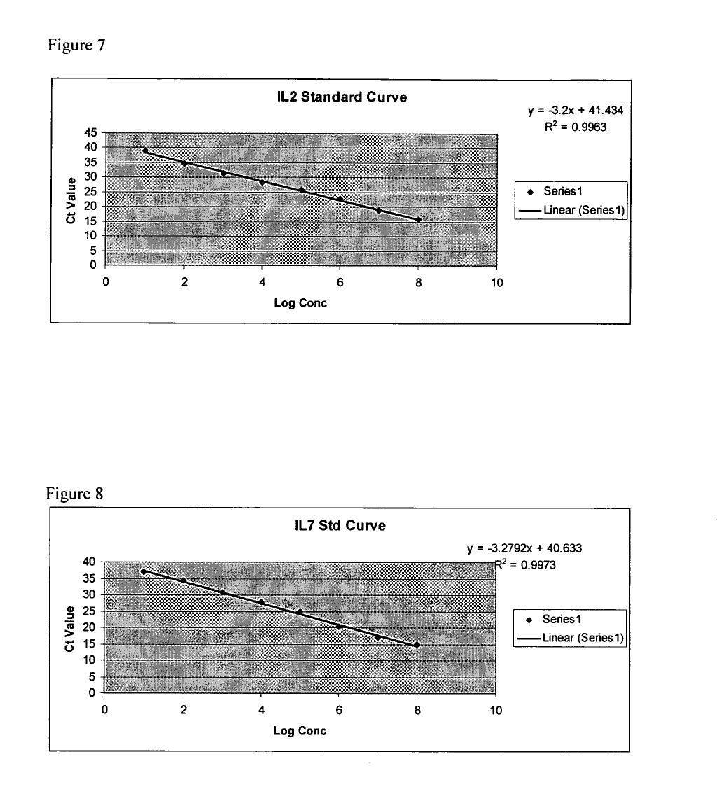

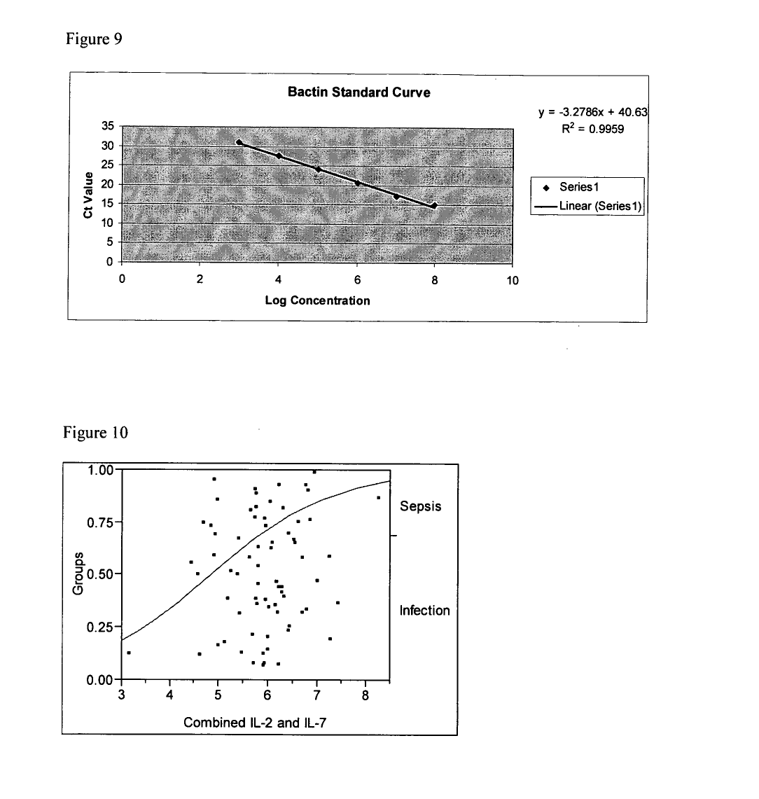

### 2.2.1 Standard Curve IL2 and IL7 and the House-Keeping Gene β-Actin. (FIGS. 7, 8 and 9)

The slope of the standard curve can be used to determine the exponential amplification a efficiency of the QRT-PCR reaction. A slope between −3.2 and −3.6 is an acceptable efficiency. The ideal QRT-PCR reaction has an efficiency of 1.0092 and amplification of 2.0092, this corresponds to a slope of −3.3.

### 3. Brief Description of Results

The results from a first cohort of patients are provided in Examples 1 to 12 below.

### Example 1 (to 12)

In an additional data set of patients with sepsis, patients with bacterial infection who did not develop sepsis and a control group, IL-2 and IL-7 mRNA levels in peripheral blood leukocytes were reduced in patients with sepsis compared to control patients and patients with bacterial infection.

Thus IL-2 mRNA copy numbers are lesser in sepsis than with infection, and are lesser in infection than in control patients.

IL-2 mRNA Copy Numbers

| Level | Minimum | 10% | 25% | Median | 75% | 90% | Maximum |
|---|---|---|---|---|---|---|---|
| Control | 122.1559 | 211.7746 | 306.6765 | 656.4726 | 1152.576 | 1679.923 | 1682.266 |
| Infection | 16.71462 | 51.78462 | 144.302 | 199.7228 | 379.9402 | 768.6838 | 2072.529 |
| Sepsis | 16.78441 | 29.46672 | 43.98022 | 104.2151 | 178.094 | 581.2909 | 1551.134 |

### Wilcoxon/Kruskal-Wallis Tests (Rank Sums)

| | | | | (Mean − |
|---|---|---|---|---|
| Level | Count | Score Sum | Score Mean | Mean0)/Std0 |
| Control | 20 | 1615.00 | 80.7500 | 4.525 |
| Infection | 50 | 2685.00 | 53.7000 | 0.221 |
| Sepsis | 35 | 1265.00 | 36.1429 | −4.007 |

### 1-Way Test, ChiSquare Approximation

| ChiSquare | DF | Prob > ChiSq |
|---|---|---|
| 27.3547 | 2 | <.0001 |

Similarly IL-7 Copy numbers are less in patients with sepsis than infection, and are less in patients with infection than a control group.

### IL-7 Copy Numbers

| Level | Minimum | 10% | 25% | Median | 75% | 90% | Maximum |
|---|---|---|---|---|---|---|---|
| Control | 2430.134 | 3640.706 | 5317.928 | 6518.609 | 11323.74 | 13046.01 | 20571.62 |
| Infection | 27.93651 | 2364.416 | 3029.921 | 5447.709 | 8947.922 | 13438.1 | 211279.9 |
| Sepsis | 377.714 | 1087.902 | 1621.671 | 3035.628 | 5689.765 | 7978.581 | 17673.52 |

### Wilcoxon/Kruskal-Wallis Tests (Rank Sums)

| | | | | (Mean − |
|---|---|---|---|---|
| Level | Count | Score Sum | Score Mean | Mean0)/Std0 |
| Control | 19 | 1462.00 | 76.9474 | 2.806 |
| Infection | 53 | 3373.00 | 63.6415 | 1.846 |
| Sepsis | 42 | 1720.00 | 40.9524 | −4.080 |

### 1-Way Test, ChiSquare Approximation

| ChiSquare | DF | Prob > ChiSq |
|---|---|---|
| 18.9341 | 2 | <.0001 |

### Example 2

The mRNA levels of these two cytokines, IL-2 and IL-7, can be combined to produce a risk score for sepsis. This score can be derived from the sum of the log to base 10 of the mRNA copy numbers.

Thus for the three groups this data shows that the combines cytokine copy numbers are reduced in patients with sepsis compared with infection, and in turn are reduced in patients with infection compared with sepsis.

### Combined IL-2 and IL-7 (Log Base 10 Copy Numbers).

| Level | Number | Mean | Std Error | Lower 95% | Upper 95% |
|---|---|---|---|---|---|
| Control | 19 | 6.59995 | 0.16547 | 6.2711 | 6.9288 |
| Infection | 49 | 6.07458 | 0.10304 | 5.8698 | 6.2794 |
| Sepsis | 22 | 5.60276 | 0.15378 | 5.2971 | 5.9084 |

### Analysis of Variance

| | | Sum of | Mean | | |
|---|---|---|---|---|---|
| Source | DF | Squares | Square | F Ratio | Prob > F |
| Groups | 2 | 10.140035 | 5.07002 | 9.7455 | 0.0002 |
| Error | 87 | 45.261060 | 0.52024 | | |
| C. Total | 89 | 55.401095 | | | |

### Means Comparisons

### Comparisons for all Pairs Using Tukey-Kramer HSD

| Abs(Dif) − LSD | Control | Infection | Sepsis |
|---|---|---|---|
| Control | −0.55800 | 0.06055 | 0.45855 |
| Infection | 0.06055 | −0.34747 | 0.03044 |
| Sepsis | 0.45855 | 0.03044 | −0.51856 |

Positive values show pairs of means that are significantly different.

| Level | | | | Mean |
|---|---|---|---|---|
| Control | A | | | 6.5999473 |
| Infection | | B | | 6.0745817 |
| Sepsis | | | C | 5.6027562 |

Levels not connected by same letter are significantly different.

### Example 3

Furthermore from this scoring system a risk of developing sepsis can be derived by comparing infection and sepsis groups in a logistic regression analysis (FIG. 10).

### Whole Model Test

| Model | -LogLikelihood | DF | ChiSquare | Prob > ChiSq |
|---|---|---|---|---|
| Difference | 2.756830 | 1 | 5.513659 | 0.0189 |
| Full | 41.191313 | | | |
| Reduced | 43.948143 | | | |

| | |
|---|---|
| RSquare (U) | 0.0627 |
| Observations (or Sum Wgts) | 71 |

### Parameter Estimates

| | | | | Prob > | |
|---|---|---|---|---|---|
| Term | Estimate | Std Error | ChiSquare | ChiSq | Odds Ratio |
| Intercept | −3.9083567 | 2.1455026 | 3.32 | 0.0685 | . |
| Combined | 0.80533408 | 0.367884 | 4.79 | 0.0286 | 60.19321 |
| IL-2 and | | | | | |
| IL-7 | | | | | |

Over the range of this scale the risk for sepsis changes by 60.2 fold. That is the risk for sepsis is 60.2 times greater in a patient with the least score compared with a patient with the greatest score. The risk for sepsis increases by 2.24 fold for each unit change in the score.

### Example 4

### Score A

However the mRNA copy numbers of the cytokines IL-2 and IL-7 can be incorporated, individually into scoring systems based on mRNA copy numbers of the cytokines IL-23, IL-10 and Interferon Gamma.

In these scoring systems the log base 10 of the cytokine mRNA copy numbers are calculated. These values are the actual corrected read out from the PCR runs.

In this algorithm the values for IL-2 or IL-7 are added to the value for IL-23 and Interferon gamma. This result is then subtracted from the value for IL-10.

In this manner the values for pro inflammatory cytokines, IL-2, IL-7, IL-23 and interferon gamma are normalised to the value for an anti inflammatory cytokine such as IL-10.

### Thus Consider the Combination of IL-7, IL-23, Interferon Gamma, and IL-10. Referred to as Summated Score A.

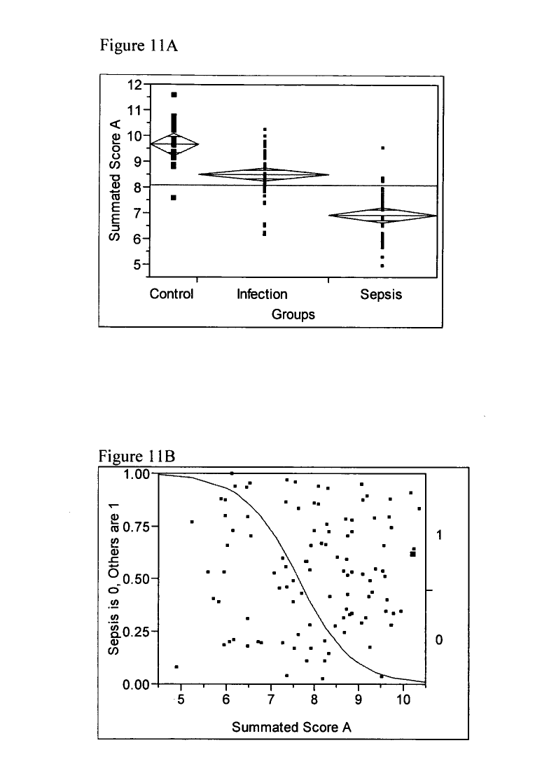

This score is greater in controls than in patients with infection, and greater in patients with infection than in patients with Sepsis (FIG. 11A).

| Level | Number | Mean | Std Error | Lower 95% | Upper 95% |
|---|---|---|---|---|---|
| Control | 18 | 9.65626 | 0.22228 | 9.2156 | 10.097 |
| Infection | 50 | 8.49983 | 0.13337 | 8.2354 | 8.764 |
| Sepsis | 41 | 6.90635 | 0.14728 | 6.6143 | 7.198 |

### Analysis of Variance

| | | Sum of | Mean | | |
|---|---|---|---|---|---|
| Source | DF | Squares | Square | F Ratio | Prob > F |
| Groups | 2 | 109.99705 | 54.9985 | 61.8388 | <.0001 |
| Error | 106 | 94.27484 | 0.8894 | | |

| Level | | | | Mean |
|---|---|---|---|---|
| Control | A | | | 9.6562613 |
| Infection | | B | | 8.4998250 |
| Sepsis | | | C | 6.9063481 |

Levels not connected by same letter are significantly different.

The risk for developing sepsis in patients with infection can be estimated with this score by using logistic regression analysis.

In this scoring system, as the rated score increases the risk for sepsis is reduced (FIG. 11B).

### Whole Model Test

| Model | -LogLikelihood | DF | ChiSquare | Prob > ChiSq |
|---|---|---|---|---|
| Difference | 22.683792 | 1 | 45.36758 | <.0001 |
| Full | 39.946818 | | | |
| Reduced | 62.630610 | | | |

| | |
|---|---|
| RSquare (U) | 0.3622 |
| Observations (or Sum Wgts) | 91 |
| Converged by Gradient | |

### Parameter Estimates

| Term | Estimate | Std Error | ChiSquare | Prob > ChiSq |
|---|---|---|---|---|
| Intercept | 12.240006 | 2.5639555 | 22.79 | <.0001 |
| Summated | −1.6049533 | 0.3279525 | 23.95 | <.0001 |
| Score B | | | | |

The Odds ratio per unit change is as follows.

For unit increase in the score the risk for sepsis increases by 0.2 fold, that is it decreases 5 fold.

Over the range of the scoring system the overall risk of sepsis increases 5000 fold form highest score with the least risk, to lowest score with the greatest risk.

### Example 5

### Score B

Now consider summated scoring system which includes IL-2, IL-23, Interferon Gamma and IL-10.

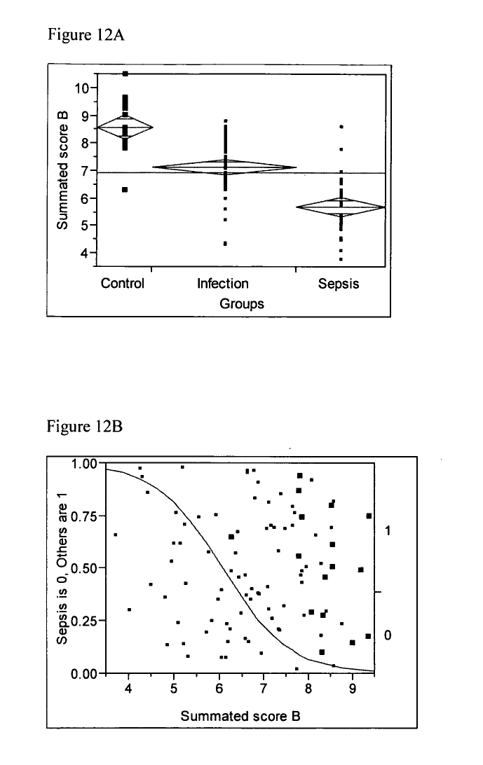

This summated score, score B is clearly different in the three groups (FIG. 12A).

| Level | Number | Mean | Std Error | Lower 95% | Upper 95% |
|---|---|---|---|---|---|
| Control | 19 | 8.55940 | 0.22431 | 8.1141 | 9.0047 |
| Infection | 49 | 7.10814 | 0.13968 | 6.8309 | 7.3854 |
| Sepsis | 31 | 5.67185 | 0.17561 | 5.3233 | 6.0204 |

### Analysis of Variance

| | | Sum of | Mean | | |
|---|---|---|---|---|---|
| Source | DF | Squares | Square | F Ratio | Prob > F |
| Groups | 2 | 101.06520 | 50.5326 | 52.8590 | <.0001 |
| Error | 96 | 91.77488 | 0.9560 | | |
| C. Total | 98 | 192.84007 | | | |

The risk for developing sepsis in patients with infection can be estimated with this score by using logistic regression analysis.

In this scoring system, as the rated score increases the risk for sepsis is reduced (Figure Example 12B).

### Whole Model Test

| Model | -LogLikelihood | DF | ChiSquare | Prob > ChiSq |
|---|---|---|---|---|
| Difference | 15.591304 | 1 | 31.18261 | <.0001 |
| Full | 37.818028 | | | |
| Reduced | 53.409333 | | | |

| | |
|---|---|
| RSquare (U) | 0.2919 |
| Observations (or Sum Wgts) | 80 |

| Term | Estimate | Std Error | ChiSquare | Prob > ChiSq |
|---|---|---|---|---|
| Intercept | 8.32746821 | 2.0157838 | 17.07 | <.0001 |
| Summated | −1.3685629 | 0.3136444 | 19.04 | <.0001 |
| score B | | | | |

In this system as the score decreased by each unit, the risk for sepsis increased 4 fold.

Over the range of the score, as the score decreased, the risk of sepsis increased by 2500 fold, i.e. when the risk associated with the highest score was compared with the risk associated with the lowest score.

### Example 6

### Score C

Additional cytokines can be added to this algorithm. The best discrimination between patients with sepsis and infection is with a combination of IL-7, IL-23, Interferon gamma on one hand and IL-10 and IL-27.

This score, Summated Score C is different in control, Infection and Sepsis groups (Figure Example 13A).

| | |
|---|---|
| Rsquare | 0.575831 |
| Adj Rsquare | 0.567752 |
| Root Mean Square Error | 1.023602 |
| Mean of Response | 5.078865 |
| Observations (or Sum Wgts) | 108 |

| | | Sum of | Mean | | |
|---|---|---|---|---|---|
| Source | DF | Squares | Square | F Ratio | Prob > F |
| Groups | 2 | 149.35089 | 74.6754 | 71.2714 | <.0001 |
| Error | 105 | 110.01495 | 1.0478 | | |
| C. Total | 107 | 259.36584 | | | |

### Means for Oneway Anova

| Level | Number | Mean | Std Error | Lower 95% | Upper 95% |
|---|---|---|---|---|---|
| Control | 17 | 7.11602 | 0.24826 | 6.6238 | 7.6083 |
| Infection | 50 | 5.46727 | 0.14476 | 5.1802 | 5.7543 |
| Sepsis | 41 | 3.76053 | 0.15986 | 3.4436 | 4.0775 |

The risk for developing sepsis in patients with infection can be estimated with this score by using logistic regression analysis.

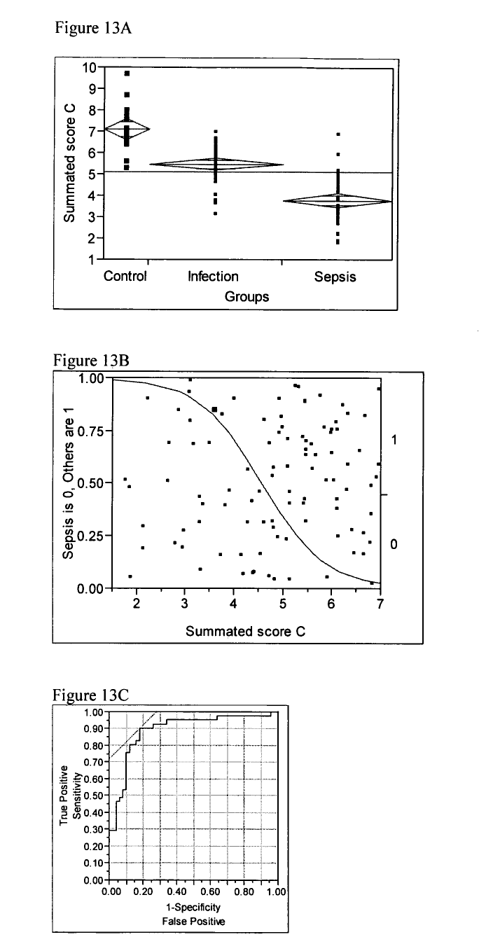

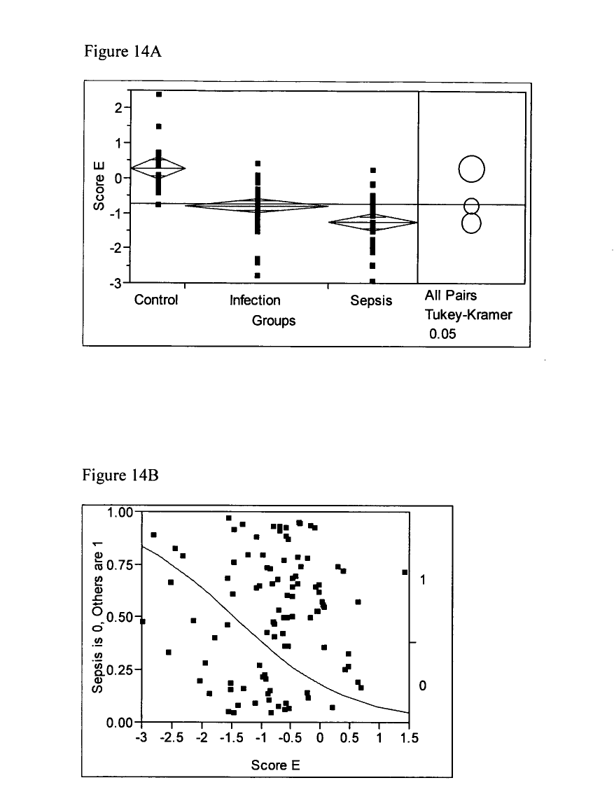

In this scoring system, as the rated score increases the risk for sepsis is reduced (FIG. 13B).

| Model | -LogLikelihood | DF | ChiSquare | Prob > ChiSq |
|---|---|---|---|---|
| Difference | 23.020230 | 1 | 46.04046 | <.0001 |
| Full | 39.610381 | | | |
| Reduced | 62.630610 | | | |

| | |
|---|---|
| RSquare (U) | 0.3676 |
| Observations (or Sum Wgts) | 91 |

### Parameter Estimates

| | | | | | Unit Odds | Odds |
|---|---|---|---|---|---|---|
| Term | Estimate | Std Error | ChiSquare | Prob > ChisSq | Ratio | Ratio |
| Intercept | 7.07811881 | 1.5455212 | 20.97 | <.0001 | — | — |
| Summated score | −1.5482397 | 0.3197876 | 23.44 | <.0001 | 0.21262192 | 0.00031881 |
| C | | | | | | |

In this system as the score decreased by each unit, the risk for sepsis increased approximately fold.

Over the range of the score, as the score decreased, the risk of sepsis increased by 3300 fold, i.e. when the risk associated with the highest score was compared with the risk associated with the lowest score.

This scoring system had an ROC curve as follows (FIG. 13C). Area Under Curve=0.88927

### Example 7

### Score E

IL-2 copy numbers can be combined with each of IL-10 and Interferon gamma and IL-23 mRNA copy numbers to produce a summated scoring system. In this method the Log to base 10 of copy numbers are added in the case of IL-2 and IL-23 and interferon gamma, or subtracted as in the case of IL-2 and IL-10.

The difference between IL-2 and Il-10 mRNA copy numbers to base 10.

This scoring system differentiates between controls, patients with Infection and patients with Sepsis: with a lower score in patients with infection compared to controls, and a lower score in patients with sepsis compared to patients with infection (FIG. 14A).

### Analysis of Variance

| | | Sum of | Mean | | |
|---|---|---|---|---|---|
| Source | DF | Squares | Square | F Ratio | Prob > F |
| Groups | 2 | 28.445782 | 14.2229 | 31.2802 | <.0001 |
| Error | 97 | 44.105204 | 0.4547 | | |
| C. Total | 99 | 72.550986 | | | |

### Means for Oneway Anova

| Level | Number | Mean | Std Error | Lower 95% | Upper 95% |
|---|---|---|---|---|---|
| Control | 19 | 0.2838 | 0.15470 | −0.023 | 0.591 |
| Infection | 50 | −0.7887 | 0.09536 | −0.978 | −0.599 |
| Sepsis | 31 | −1.2612 | 0.12111 | −1.502 | −1.021 |

### Means Comparisons

### Comparisons for all Pairs Using Tukey-Kramer HSD

| q* | Alpha |
|---|---|
| 2.38024 | 0.05 |

| Abs(Dif) − LSD | Control | Infection | Sepsis |
|---|---|---|---|
| Control | −0.5207 | 0.6399 | 1.0773 |
| Infection | 0.6399 | −0.3210 | 0.1056 |
| Sepsis | 1.0773 | 0.1056 | −0.4077 |

Positive values show pairs of means that are significantly different.

| Level | | | | Mean |
|---|---|---|---|---|
| Control | A | | | 0.283774 |
| Infection | | B | | −0.788677 |
| Sepsis | | | C | −1.261182 |

Levels not connected by same letter are significantly different.

This score, SCORE E, can be used in a logistic regression analysis to determine the risk for sepsis in patients with infection (FIG. 14B).

### Whole Model Test

| Model | -LogLikelihood | DF | ChiSquare | Prob > ChiSq |
|---|---|---|---|---|
| Difference | 4.500243 | 1 | 9.000487 | 0.0027 |
| Full | 49.395384 | | | |
| Reduced | 53.895628 | | | |

| | |
|---|---|
| RSquare (U) | 0.0835 |
| Observations (or Sum Wgts) | 81 |

| Term | Estimate | Std Error | ChiSquare | Prob > ChiSq |
|---|---|---|---|---|
| Intercept | −1.5240247 | 0.4509522 | 11.42 | 0.0007 |
| Score E | −1.0416064 | 0.3744524 | 7.74 | 0.0054 |

| Unit Odds Ratio | Odds Ratio |
|---|---|
| 0.35288733 | 0.01038771 |

Thus as the score increases the risk for sepsis decreases. Patients with the greatest score have 0.01 times the risk of sepsis as patients with the least score. For each unit increase in SCORE E the risk for sepsis decreased by 0.35, or approximately by a third. Conversely a unit decrease in SCORE E was associated with an approximate 2.8 fold increase in risk for sepsis in patients with infection, while the risk for sepsis in patients with infection was approximately 96 times greater in patients with the least score compared with patients with the greatest score.

### Example 8

### Score F

This score system is based on the sum of IL-2 and Interferon mRNA copy numbers to the base 10.

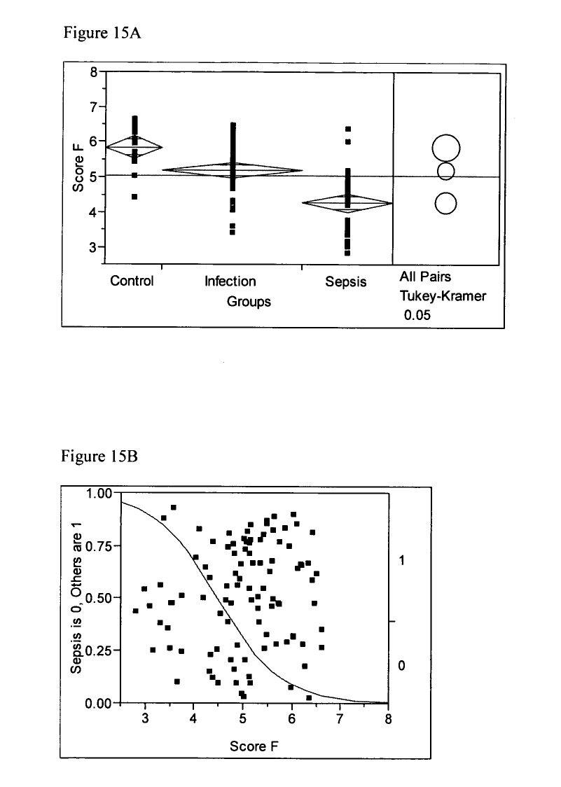

This scoring system differentiates between controls, patients with Infection and patients with Sepsis: with a lower score in patients with infection compared to controls, and a lower score in patients with sepsis compared to patients with infection (FIG. 15A).

### Analysis of Variance

| | | Sum of | Mean | | |
|---|---|---|---|---|---|
| Source | DF | Squares | Square | F Ratio | Prob > F |
| Groups | 2 | 33.341650 | 16.6708 | 31.4574 | <.0001 |
| Error | 98 | 51.934979 | 0.5299 | | |
| C. Total | 100 | 85.276629 | | | |

### Means for Oneway Anova

| Level | Number | Mean | Std Error | Lower 95% | Upper 95% |
|---|---|---|---|---|---|
| Control | 20 | 5.84048 | 0.16278 | 5.5175 | 6.1635 |
| Infection | 49 | 5.18889 | 0.10400 | 4.9825 | 5.3953 |
| Sepsis | 32 | 4.26004 | 0.12869 | 4.0047 | 4.5154 |

### Means Comparisons

### Comparisons for all Pairs Using Tukey-Kramer HSD

| q* | Alpha |
|---|---|
| 2.37986 | 0.05 |

| Abs(Dif) − LSD | Control | Infection | Sepsis |
|---|---|---|---|
| Control | −0.5479 | 0.1919 | 1.0866 |
| Infection | 0.1919 | −0.3500 | 0.5351 |
| Sepsis | 1.0866 | 0.5351 | −0.4331 |

Positive values show pairs of means that are significantly different.

| Level | | | | Mean |
|---|---|---|---|---|
| Control | A | | | 5.8404839 |
| Infection | | B | | 5.1888890 |
| Sepsis | | | C | 4.2600360 |

Levels not connected by same letter are significantly different.

This score, SCORE F, can be used in a logistic regression analysis to determine the risk for sepsis in patients with infection (FIG. 15B).

### Whole Model Test

| Model | -LogLikelihood | DF | ChiSquare | Prob > ChiSq |
|---|---|---|---|---|
| Difference | 12.095017 | 1 | 24.19003 | <.0001 |
| Full | 42.252621 | | | |
| Reduced | 54.347638 | | | |

| | |
|---|---|
| RSquare (U) | 0.2225 |
| Observations (or Sum Wgts) | 81 |

### Parameter Estimates

| Term | Estimate | Std Error | ChiSquare | Prob > ChiSq |
|---|---|---|---|---|
| Intercept | 6.90303226 | 1.8259727 | 14.29 | 0.0002 |
| Score F | −1.5419465 | 0.3814791 | 16.34 | <.0001 |

| Unit Odds Ratio | Odds Ratio |
|---|---|
| 0.21396421 | 0.00067978 |

Thus as the score increases, the risk for sepsis decreases. Patients with the greatest score have 0.0006 times (6 ten thousandths, or approximately 1472 fold times less) the risk of sepsis as patients with the least score. For each unit increase in SCORE F the risk for sepsis decreased by 0.21, or to approximately one fifth of the prior risk. Conversely a unit decrease in SCORE F was associated with approximately a five-fold increase in risk for sepsis in patients with infection, while the risk for sepsis in patients with infection was approximately 1472 times greater in patients with the least score compared with patients with the greatest score.

### Example 9

### Score G

This score system is based on the sum of IL-2 and IL-23 mRNA copy numbers to the base 10.

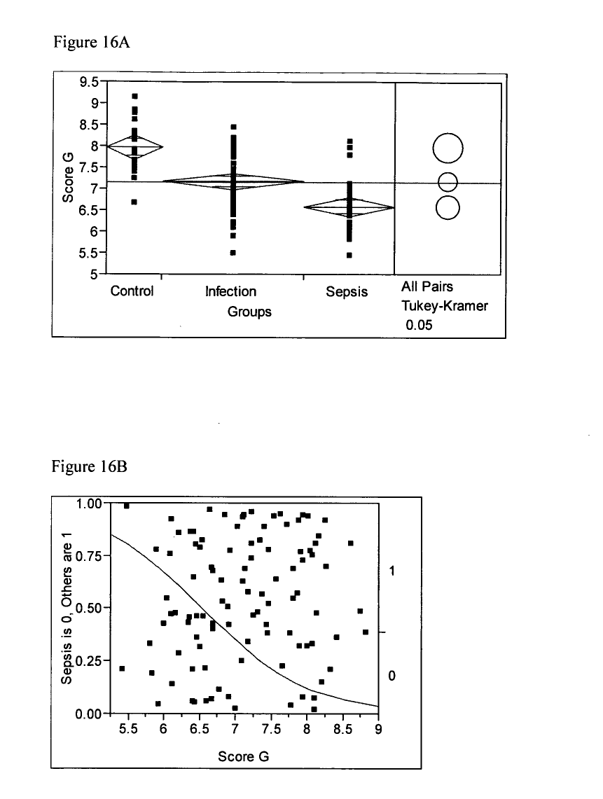

This scoring system differentiates between controls, patients with Infection and patients with Sepsis: with a lower score in patients with infection compared to controls, and a lower score in patients with sepsis compared to patients with infection (FIG. 16A).

### Analysis of Variance

| | | Sum of | Mean | | |
|---|---|---|---|---|---|
| Source | DF | Squares | Square | F Ratio | Prob > F |
| Groups | 2 | 24.016167 | 12.0081 | 27.9328 | <.0001 |
| Error | 99 | 42.559324 | 0.4299 | | |
| C. Total | 101 | 66.575491 | | | |

### Means for Oneway Anova

| Level | Number | Mean | Std Error | Lower 95% | Upper 95% |
|---|---|---|---|---|---|
| Control | 20 | 7.96964 | 0.14661 | 7.6787 | 8.2605 |
| Infection | 50 | 7.17054 | 0.09272 | 6.9866 | 7.3545 |
| Sepsis | 32 | 6.57532 | 0.11591 | 6.3453 | 6.8053 |

### Means Comparisons

### Comparisons for all Pairs Using Tukey-Kramer HSD

| q* | Alpha |
|---|---|
| 2.37950 | 0.05 |

| Abs(Dif) − LSD | Control | Infection | Sepsis |
|---|---|---|---|
| Control | −0.49336 | 0.38632 | 0.94961 |
| Infection | 0.38632 | −0.31203 | 0.24202 |
| Sepsis | 0.94961 | 0.24202 | −0.39004 |

Positive values show pairs of means that are significantly different.

| Level | | | | Mean |
|---|---|---|---|---|
| Control | A | | | 7.9696387 |
| Infection | | B | | 7.1705385 |
| Sepsis | | | C | 6.5753222 |

Levels not connected by same letter are significantly different.

This score, SCORE G, can be used in a logistic regression analysis to determine the risk for sepsis in patients with infection (FIG. 16B).

### Whole Model Test

| Model | -LogLikelihood | DF | ChiSquare | Prob > ChiSq |
|---|---|---|---|---|
| Difference | 7.203572 | 1 | 14.40714 | 0.0001 |
| Full | 47.642707 | | | |
| Reduced | 54.846279 | | | |

| | |
|---|---|
| RSquare (U) | 0.1313 |
| Observations (or Sum Wgts) | 82 |

### Parameter Estimates

| Term | Estimate | Std Error | ChiSquare | Prob > ChiSq |
|---|---|---|---|---|
| Intercept | 8.79047124 | 2.7130131 | 10.50 | 0.0012 |
| Score G | −1.3463521 | 0.3980357 | 11.44 | 0.0007 |

| Unit Odds Ratio | Odds Ratio |
|---|---|
| 0.26018768 | 0.01036251 |

Thus as the score increases, the risk for sepsis decreases. Patients with the greatest score have 0.01 times (one hundredth) the risk of sepsis as patients with the least score. For each unit increase in SCORE G the risk for sepsis decreased by 0.26, or to approximately one quarter of the prior risk. Conversely a unit decrease in SCORE G was associated with an approximate four-fold increase in risk for sepsis in patients with infection, while the risk for sepsis in patients with infection was approximately 100 times greater in patients with the least score compared with patients with the greatest score.

### Example 10

### Score H

This score system is based on the difference between IL-7 and IL-10 mRNA copy numbers to the base 10.

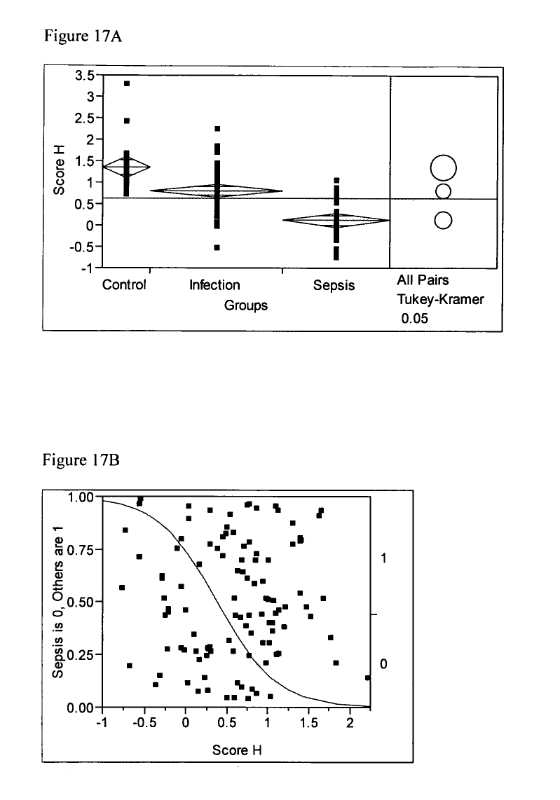

This scoring system differentiates between controls, patients with Infection and patients with Sepsis: with a lower score in patients with infection compared to controls, and a lower score in patients with sepsis compared to patients with infection (FIG. 17A).

| | |
|---|---|
| Rsquare | 0.41947 |
| Adj Rsquare | 0.408619 |
| Root Mean Square Error | 0.526087 |
| Mean of Response | 0.637382 |
| Observations (or Sum Wgts) | 110 |

### Analysis of Variance

| | | Sum of | Mean | | |
|---|---|---|---|---|---|
| Source | DF | Squares | Square | F Ratio | Prob > F |
| Groups | 2 | 21.398124 | 10.6991 | 38.6572 | <.0001 |
| Error | 107 | 29.614169 | 0.2768 | | |
| C. Total | 109 | 51.012293 | | | |

### Means for Oneway Anova

| Level | Number | Mean | Std Error | Lower 95% | Upper 95% |
|---|---|---|---|---|---|
| Control | 18 | 1.34295 | 0.12400 | 1.097 | 1.5888 |
| Infection | 51 | 0.80489 | 0.07367 | 0.659 | 0.9509 |
| Sepsis | 41 | 0.11926 | 0.08216 | −0.044 | 0.2821 |

### Means Comparisons

### Comparisons for all Pairs Using Tukey-Kramer HSD

| q* | Alpha |
|---|---|
| 2.37679 | 0.05 |

| Abs(Dif) − LSD | Control | Infection | Sepsis |
|---|---|---|---|
| Control | −0.41680 | 0.19525 | 0.87014 |
| Infection | 0.19525 | −0.24762 | 0.42334 |
| Sepsis | 0.87014 | 0.42334 | −0.27617 |

Positive values show pairs of means that are significantly different.

| Level | | | | Mean |
|---|---|---|---|---|
| Control | A | | | 1.3429480 |
| Infection | | B | | 0.8048855 |
| Sepsis | | | C | 0.1192622 |

This score, SCORE H, can be used in a logistic regression analysis to determine the risk for sepsis in patients with infection (FIG. 17B).

### Whole Model Test

| Model | -LogLikelihood | DF | ChiSquare | Prob > ChiSq |
|---|---|---|---|---|
| Difference | 17.686604 | 1 | 35.37321 | <.0001 |
| Full | 45.538383 | | | |
| Reduced | 63.224987 | | | |

| | |
|---|---|
| RSquare (U) | 0.2797 |
| Observations (or Sum Wgts) | 92 |

### Parameter Estimates

| Term | Estimate | Std Error | ChiSquare | Prob > ChiSq |
|---|---|---|---|---|
| Intercept | 1.06669221 | 0.37366 | 8.15 | 0.0043 |
| Score H | −2.7845425 | 0.602742 | 21.34 | <.0001 |

| Unit Odds Ratio | Odds Ratio |
|---|---|
| 0.06175734 | 0.00024141 |

Thus as the score increases the risk for sepsis decreases. Patients with the greatest score have 0.0002 times (one five thousandth) the risk of sepsis as patients with the least score. For each unit increase in SCORE H the risk for sepsis decreased by 0.06, or to one sixteenth of the prior risk. Conversely a unit decrease in SCORE H was associated with a sixteen-fold increase in risk for sepsis in patients with infection, while the risk for sepsis in patients with infection was approximately 4000 times greater in patients with the least score compared with patients with the greatest score.

### Example 11

### Score I

This score system is based on the sum of IL-7 and Interferon mRNA copy numbers to the base 10.

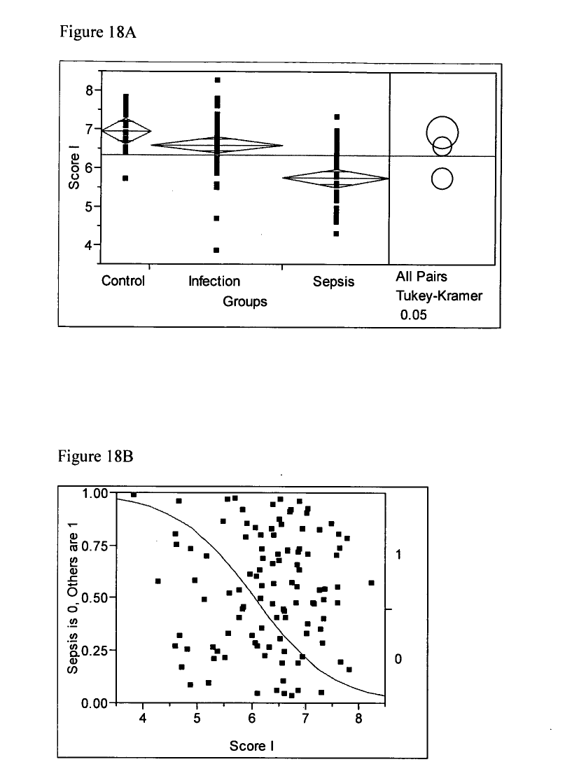

This scoring system differentiates between controls, patients with Infection and patients with Sepsis: with a lower score in patients with infection compared to controls, and a lower score in patients with sepsis compared to patients with infection (FIG. 18A).

### Analysis of Variance

| | | Sum of | Mean | | |
|---|---|---|---|---|---|
| Source | DF | Squares | Square | F Ratio | Prob > F |
| Groups | 2 | 24.715614 | 12.3578 | 21.9466 | <.0001 |
| Error | 107 | 60.250212 | 0.5631 | | |
| C. Total | 109 | 84.965826 | | | |

### Means for Oneway Anova

| Level | Number | Mean | Std Error | Lower 95% | Upper 95% |
|---|---|---|---|---|---|
| Control | 19 | 6.94545 | 0.17215 | 6.6042 | 7.2867 |
| Infection | 50 | 6.59176 | 0.10612 | 6.3814 | 6.8021 |
| Sepsis | 41 | 5.74361 | 0.11719 | 5.5113 | 5.9759 |

### Means Comparisons

### Comparisons for all Pairs Using Tukey-Kramer HSD

| q* | Alpha |
|---|---|
| 2.37679 | 0.05 |

| Abs(Dif) − LSD | Control | Infection | Sepsis |
|---|---|---|---|
| Control | −0.57865 | −0.12698 | 0.70686 |
| Infection | −0.12698 | −0.35670 | 0.47238 |
| Sepsis | 0.70686 | 0.47238 | −0.39391 |

Positive values show pairs of means that are significantly different.

| Level | | | Mean |
|---|---|---|---|
| Control | A | | 6.9454503 |
| Infection | A | | 6.5917621 |
| Sepsis | | B | 5.7436135 |

Levels not connected by same letter are significantly different.

This score, SCORE I, can be used in a logistic regression analysis to determine the risk for sepsis in patients with infection (FIG. 18B).

### Whole Model Test

| Model | -LogLikelihood | DF | ChiSquare | Prob > ChiSq |
|---|---|---|---|---|
| Difference | 11.557743 | 1 | 23.11549 | <.0001 |
| Full | 51.072867 | | | |
| Reduced | 62.630610 | | | |

| | |
|---|---|
| RSquare (U) | 0.1845 |
| Observations (or Sum Wgts) | 91 |

### Parameter Estimates

| | | | | Prob > | |
|---|---|---|---|---|---|
| Term | Estimate | Std Error | ChiSquare | ChiSq | Odds Ratio |
| Intercept | 8.28323342 | 2.1110414 | 15.40 | <.0001 | . |
| Score I | −1.3688679 | 0.3377283 | 16.43 | <.0001 | 0.00235533 |

| Unit Odds Ratio | Odds Ratio |
|---|---|
| 0.25439481 | 0.00235533 |

Thus as the score increases the risk for sepsis decreases. Patients with the greatest score have 0.002 times (one five hundredth) the risk of sepsis as patients with the least score. For each unit increase in SCORE I the risk for sepsis decreased by 0.25, or to one approximately a quarter of the prior risk. Conversely a unit decrease in SCORE I was associated with an approximate four-fold increase in risk for sepsis in patients with infection, while the risk for sepsis in patients with infection was approximately 424 times greater in patients with the least score compared with patients with the greatest score.

### Example 12

### Score J

This score system is based on the sum of IL-2 and IL-23 mRNA copy numbers to the base 10.

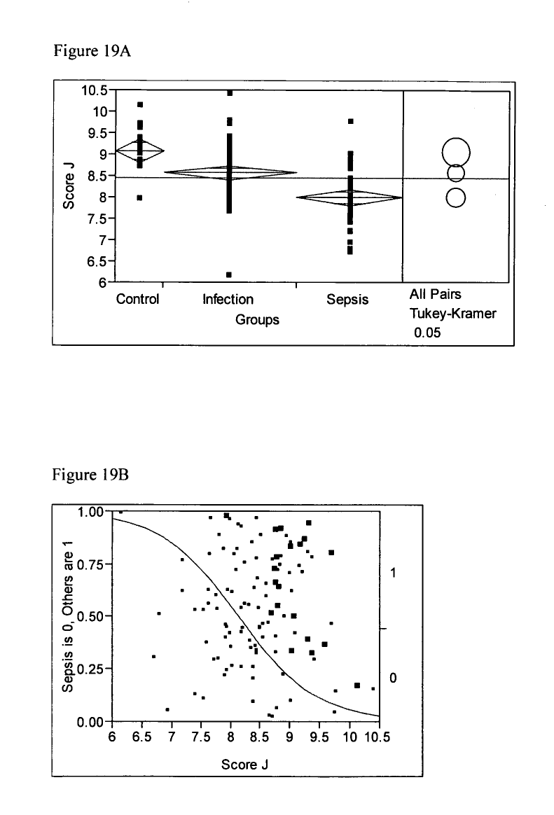

This scoring system differentiates between controls, patients with Infection and patients with Sepsis: with a lower score in patients with infection compared to controls, and a lower score in patients with sepsis compared to patients with infection (FIG. 19A).

### Analysis of Variance

| | | Sum of | Mean | | |
|---|---|---|---|---|---|
| Source | DF | Squares | Square | F Ratio | Prob > F |
| Groups | 2 | 16.559547 | 8.27977 | 22.3690 | <.0001 |
| Error | 109 | 40.345769 | 0.37014 | | |
| C. Total | 111 | 56.905316 | | | |

### Means for Oneway Anova

| Level | Number | Mean | Std Error | Lower 95% | Upper 95% |
|---|---|---|---|---|---|
| Control | 19 | 9.06886 | 0.13958 | 8.7922 | 9.3455 |
| Infection | 52 | 8.56451 | 0.08437 | 8.3973 | 8.7317 |
| Sepsis | 41 | 7.99171 | 0.09502 | 7.8034 | 8.1800 |

### Means Comparisons

### Comparisons for all Pairs Using Tukey-Kramer HSD

| q* | Alpha |
|---|---|
| 2.37618 | 0.05 |

| Abs(Dif) − LSD | Control | Infection | Sepsis |
|---|---|---|---|
| Control | −0.46903 | 0.11681 | 0.67594 |
| Infection | 0.11681 | −0.28352 | 0.27087 |
| Sepsis | 0.67594 | 0.27087 | −0.31929 |

Positive values show pairs of means that are significantly different.

| Level | | | | Mean |
|---|---|---|---|---|
| Control | A | | | 9.0688604 |
| Infection | | B | | 8.5645099 |
| Sepsis | | | C | 7.9917084 |

Levels not connected by same letter are significantly different.

This score, SCORE J, can be used in a logistic regression analysis to determine the risk for sepsis in patients with infection (FIG. 19B).

### Whole Model Test

| Model | -LogLikelihood | DF | ChiSquare | Prob > ChiSq |
|---|---|---|---|---|
| Difference | 8.743534 | 1 | 17.48707 | <.0001 |
| Full | 54.481453 | | | |
| Reduced | 63.224987 | | | |

| | |
|---|---|
| RSquare (U) | 0.1383 |
| Observations (or Sum Wgts) | 92 |

### Parameter Estimates

| Term | Estimate | Std Error | ChiSquare | Prob > ChiSq |
|---|---|---|---|---|
| Intercept | 12.5106108 | 3.5899849 | 12.14 | 0.0005 |
| Score J | −1.5373514 | 0.433562 | 12.57 | 0.0004 |

| Unit Odds Ratio | Odds Ratio |
|---|---|
| 0.21494967 | 0.00147077 |

Thus as the score increases the risk for sepsis decreases. Patients with the greatest score have 0.0014 times (approximately one seven hundredth) the risk of sepsis as patients with the least score. For each unit increase in SCORE J the risk for sepsis decreased by 0.21, or to approximately one fifth of the prior risk. Conversely a unit decrease in SCORE J was associated with an approximate five-fold increase in risk for sepsis in patients with infection, while the risk for sepsis in patients with infection was at an approximately 680 times greater in patients with the least score compared with patients with the greatest score.

Examples 13 to 24 below are carried out using a cohort of patients different to those for which Examples 1 to 12 are based. Like the first cohort of patients, the second cohort of patients comprises three distinct groups; Healthy Controls, Patients with Infection, and Patients with Severe sepsis.

### Example 13 and 14

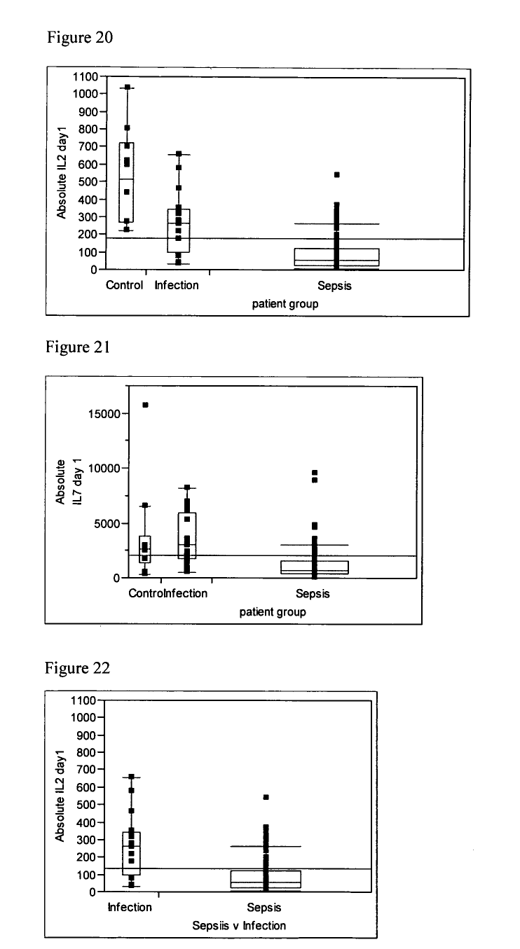

Examples 13 and 14 give the distribution of IL-2 and IL-7 mRNA levels in three groups of patients; Healthy Controls, Patients with Infection, and Patients with Severe sepsis. These two examples contain a statistical analysis of the comparison between the three groups and relate to FIGS. 20 and 21.

### Example 13

### IL-2 mRNA Levels in Three Patient Groups

### Quantiles

| Level | Minimum | 10% | 25% | Median | 75% | 90% | Maximum |
|---|---|---|---|---|---|---|---|
| Control | 222.7601 | 227.2838 | 270.8055 | 514.5454 | 722.5639 | 1006.729 | 1029.959 |
| Infection | 29.92869 | 30.5752 | 97.44937 | 264.6081 | 342.9062 | 596.98 | 653.0492 |
| Severe | 4.513578 | 16.07357 | 23.10027 | 55.99495 | 120.2476 | 281.0151 | 535.4691 |
| sepsis | | | | | | | |

### Wilcoxon/Kruskal-Wallis Tests (Rank Sums)

| | | | | (Mean − |
|---|---|---|---|---|
| Level | Count | Score Sum | Score Mean | Mean0)/Std0 |
| Control | 10 | 796.000 | 79.6000 | 4.372 |
| Infection | 16 | 974.000 | 60.8750 | 2.591 |
| Severe sepsis | 64 | 2325.00 | 36.3281 | −5.221 |

### 1-Way Test, ChiSquare Approximation

| ChiSquare | DF | Prob > ChiSq |
|---|---|---|
| 30.4678 | 2 | <.0001 |

### Example 14

### IL-7 mRNA Levels in Three Patient Groups

### Quantiles

| Level | Minimum | 10% | 25% | Median | 75% | 90% | Maximum |
|---|---|---|---|---|---|---|---|
| Control | 283.0553 | 300.9931 | 1372.122 | 2658.712 | 3819.715 | 14730.06 | 15641.59 |
| Infection | 487.0186 | 753.2133 | 1791.384 | 3008.941 | 5924.231 | 7239.475 | 8124.894 |
| Severe | 38.02993 | 172.5917 | 407.9582 | 737.0873 | 1587.198 | 3218.928 | 211242 |
| sepsis | | | | | | | |

### Wilcoxon/Kruskal-Wallis Tests (Rank Sums)

| | | | | (Mean − |
|---|---|---|---|---|
| Level | Count | Score Sum | Score Mean | Mean0)/Std0 |
| Control | 10 | 617.000 | 61.7000 | 1.986 |
| Infection | 16 | 1090.00 | 68.1250 | 3.685 |
| Severe sepsis | 65 | 2479.00 | 38.1385 | −4.485 |

### 1-Way Test, ChiSquare Approximation

| ChiSquare | DF | Prob > ChiSq |
|---|---|---|
| 20.5175 | 2 | <.0001 |

### Examples 15 and 16

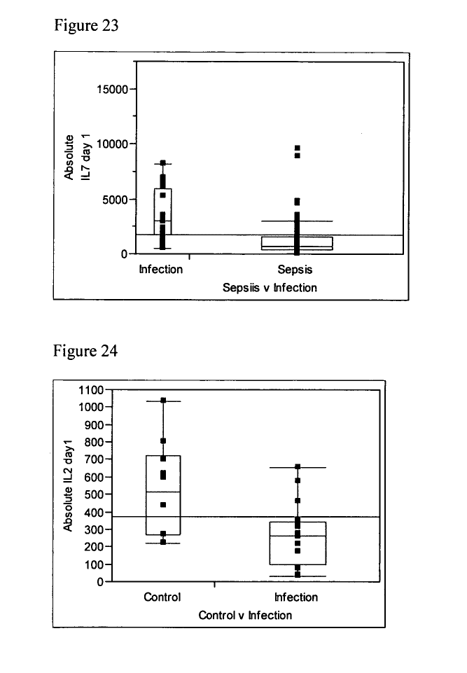

Examples 15 and 16 give the distribution of IL-2 and IL-7 mRNA levels in two groups of patients; Patients with Infection, and Patients with Severe sepsis. These two examples contain statistical analyses of the comparison between the three groups and relate to FIGS. 22 and 23.

### Example 15

### IL-2 mRNA Levels in Two Patient Groups; Infection and Severe Sepsis

### Quantiles

| Level | Minimum | 10% | 25% | Median | 75% | 90% | Maximum |
|---|---|---|---|---|---|---|---|
| Infection | 29.92869 | 30.5752 | 97.44937 | 264.6081 | 342.9062 | 596.98 | 653.0492 |
| Severe | 4.513578 | 16.07357 | 23.10027 | 55.99495 | 120.2476 | 281.0151 | 535.4691 |
| sepsis | | | | | | | |

### Wilcoxon/Kruskal-Wallis Tests (Rank Sums)

| | | | | (Mean − |
|---|---|---|---|---|
| Level | Count | Score Sum | Score Mean | Mean0)/Std0 |
| Infection | 16 | 941.000 | 58.8125 | 3.518 |
| Severe sepsis | 64 | 2299.00 | 35.9219 | −3.518 |

### 2-Sample Test, Normal Approximation

| S | Z | Prob > \|Z\| |
|---|---|---|
| 941 | 3.51823 | 0.0004 |

### 1-Way Test, ChiSquare Approximation

| ChiSquare | DF | Prob > ChiSq |
|---|---|---|
| 12.4203 | 1 | 0.0004 |

### Example 16

### IL-7 mRNA Levels in Two Patient Groups; Infection and Severe Sepsis

### Quantiles

| Level | Minimum | 10% | 25% | Median | 75% | 90% | Maximum |
|---|---|---|---|---|---|---|---|
| Infection | 487.0186 | 753.2133 | 1791.384 | 3008.941 | 5924.231 | 7239.475 | 8124.894 |
| Severe | 38.02993 | 168.3913 | 400.7392 | 727.2953 | 1531.579 | 2875.682 | 9531.648 |
| sepsis | | | | | | | |

### Wilcoxon/Kruskal-Wallis Tests (Rank Sums)

| | | | | (Mean − |
|---|---|---|---|---|
| Level | Count | Score Sum | Score Mean | Mean0)/Std0 |
| Infection | 16 | 998.000 | 62.3750 | 4.204 |
| Severe sepsis | 64 | 2242.00 | 35.0313 | −4.204 |

### 2-Sample Test, Normal Approximation

| S | Z | Prob > \|Z\| |
|---|---|---|
| 998 | 4.20383 | <.0001 |

### 1-Way Test, ChiSquare Approximation

| ChiSquare | DF | Prob > ChiSq |
|---|---|---|
| 17.7228 | 1 | <.0001 |

### Examples 17 and 18

Examples 17 and 18 give the distribution of IL-2 and IL-7 mRNA levels in two groups of patients; Healthy Controls and Patients with Infection. These two examples contain a statistical analysis of the comparison between the three groups.

### Example 17

### IL-2 mRNA Levels in Two Patient Groups; Infection and Control (FIG. 24)

### Quantiles

| Level | Minimum | 10% | 25% | Median | 75% | 90% | Maximum |
|---|---|---|---|---|---|---|---|
| Con- | 222.7601 | 227.2838 | 270.8055 | 514.5454 | 722.5639 | 1006.729 | 1029.959 |
| trol | | | | | | | |
| Infec- | 29.92869 | 30.5752 | 97.44937 | 264.6081 | 342.9062 | 596.98 | 653.0492 |
| tion | | | | | | | |

### Wilcoxon/Kruskal-Wallis Tests (Rank Sums)

| | | | | (Mean − |
|---|---|---|---|---|
| Level | Count | Score Sum | Score Mean | Mean0)/Std0 |
| Control | 10 | 182.000 | 18.2000 | 2.451 |
| Infection | 16 | 169.000 | 10.5625 | −2.451 |

### 2-Sample Test, Normal Approximation

| S | Z | Prob > \|Z\| |
|---|---|---|
| 182 | 2.45077 | 0.0143 |

### 1-Way Test, ChiSquare Approximation

| ChiSquare | DF | Prob > ChiSq |
|---|---|---|
| 6.1361 | 1 | 0.0132 |

### Example 18

### IL-7 mRNA Levels in Two Patient Groups; Infection and Severe Sepsis

### Quantiles

| Level | Minimum | 10% | 25% | Median | 75% | 90% | Maximum |
|---|---|---|---|---|---|---|---|
| Control | 283.0553 | 300.9931 | 1372.122 | 2658.712 | 3819.715 | 14730.06 | 15641.59 |
| Infection | 487.0186 | 753.2133 | 1791.384 | 3008.941 | 5924.231 | 7239.475 | 8124.894 |

### Wilcoxon/Kruskal-Wallis Tests (Rank Sums)

| | | | | (Mean − |
|---|---|---|---|---|
| Level | Count | Score Sum | Score Mean | Mean0)/Std0 |
| Control | 10 | 123.000 | 12.3000 | −0.606 |
| Infection | 16 | 228.000 | 14.2500 | 0.606 |

### 2-Sample Test, Normal Approximation

| S | Z | Prob > \|Z\| |
|---|---|---|
| 123 | −0.60610 | 0.5444 |

### 1-Way Test, ChiSquare Approximation

| ChiSquare | DF | Prob > ChiSq |
|---|---|---|
| 0.4000 | 1 | 0.5271 |

### Example 19

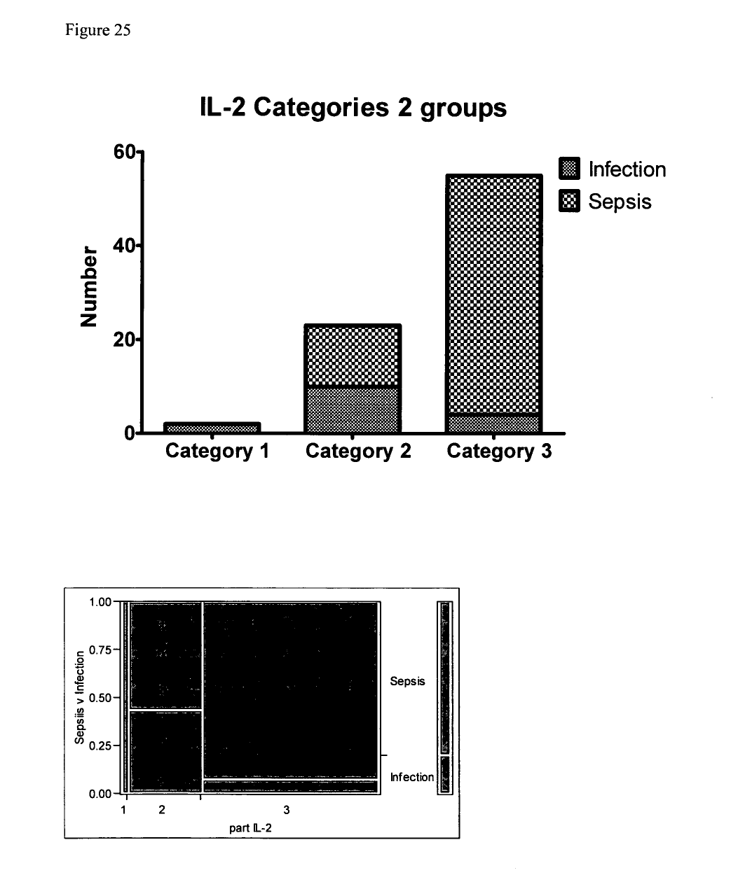

Example 19 gives the distribution of the IL-2 categories, 1 or 2 or 3, in two groups of patients; Patients with Infection, and Patients with Severe sepsis. This table contains a statistical analysis of the distribution of IL-2 categories between the two groups and relates to FIG. 25.

IL-2 levels have been divided into three levels, indicated by the numerals 1, 2 and 3.

The distribution of these IL-2 levels in patients with Infection and Severe sepsis is listed in the table.

Contingency Table

| Count | | | |
|---|---|---|---|
| Total % | | | |
| Col % | | | |
| Row % | Infection | Severe sepsis | |
| 1 | 2 | 0 | 2 |
| | 2.50 | 0.00 | 2.50 |
| | 12.50 | 0.00 | |
| | 100.00 | 0.00 | |
| 2 | 10 | 13 | 23 |
| | 12.50 | 16.25 | 28.75 |
| | 62.50 | 20.31 | |
| | 43.48 | 56.52 | |
| 3 | 4 | 51 | 55 |
| | 5.00 | 63.75 | 68.75 |
| | 25.00 | 79.69 | |
| | 7.27 | 92.73 | |
| | 16 | 64 | 80 |
| | 20.00 | 80.00 | |

### Tests

| N | DF | -LogLike | RSquare (U) |
|---|---|---|---|
| 80 | 2 | 9.9509790 | 0.2486 |

| Test | ChiSquare | Prob > ChiSq |
|---|---|---|
| Likelihood | 19.902 | <.0001 |
| Ratio | 21.492 | <.0001 |
| Pearson | | |

### Example 20

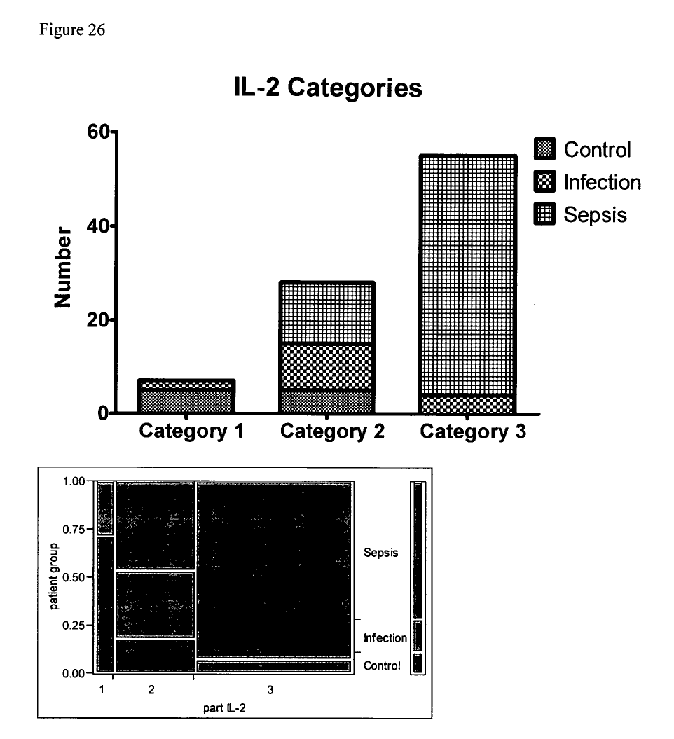

Example 20 gives the distribution of the IL-2 categories, 1 or 2 or 3, in three groups of patients; Healthy Controls, Patients with Infection, and Patients with Severe sepsis. This table contains a statistical analysis of the distribution of IL-2 categories between the three groups and relates to FIG. 26.

IL-2 levels have been divided into three levels, indicated by the numerals 1, 2 and 3.

The distribution of these IL-2 levels in a control Group and in patients with Infection and Severe sepsis is listed in the table.

| Count | | | | |
|---|---|---|---|---|
| Total % | | | | |
| Col % | | | | |
| Row % | Control | Infection | Severe sepsis | |
| 1 | 5 | 2 | 0 | 7 |
| | 5.56 | 2.22 | 0.00 | 7.78 |
| | 50.00 | 12.50 | 0.00 | |
| | 71.43 | 28.57 | 0.00 | |
| 2 | 5 | 10 | 13 | 28 |
| | 5.56 | 11.11 | 14.44 | 31.11 |
| | 50.00 | 62.50 | 20.31 | |
| | 17.86 | 35.71 | 46.43 | |
| 3 | 0 | 4 | 51 | 55 |
| | 0.00 | 4.44 | 56.67 | 61.11 |
| | 0.00 | 25.00 | 79.69 | |
| | 0.00 | 7.27 | 92.73 | |
| | 10 | 16 | 64 | 90 |
| | 11.11 | 17.78 | 71.11 | |

### Tests

| N | DF | -LogLike | RSquare (U) |
|---|---|---|---|
| 90 | 4 | 24.019811 | 0.3363 |

| Test | ChiSquare | Prob > ChiSq |
|---|---|---|
| Likelihood | 48.040 | <.0001 |
| Ratio | | |
| Pearson | 50.109 | <.0001 |

### Example 21

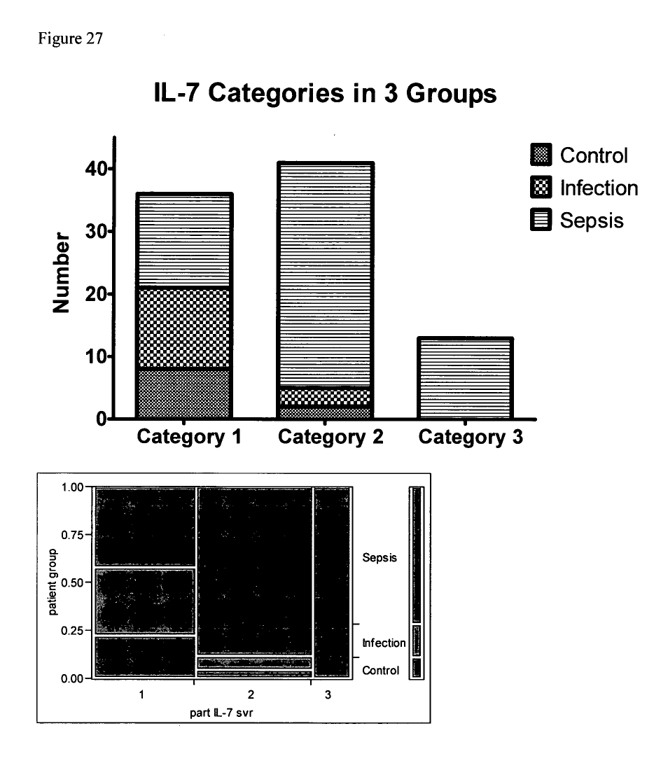

Example 21 gives the distribution of the IL-7 categories, 1 or 2 or 3, in three groups of patients; Healthy Controls, Patients with Infection, and Patients with Severe sepsis. This example contains a statistical analysis of the distribution of IL-7 categories between the three groups and relates to FIG. 27.

IL-7 levels have been divided into three levels, indicated by the numerals 1, 2 and 3.

The distribution of these IL-2 levels in a control Group and in patients with Infection and Severe sepsis is listed in the table.

| Count | | | | |
|---|---|---|---|---|
| Total % | | | | |
| Col % | | | | |
| Row % | Control | Infection | Severe sepsis | |
| 1 | 8 | 13 | 15 | 36 |
| | 8.89 | 14.44 | 16.67 | 40.00 |
| | 80.00 | 81.25 | 23.44 | |
| | 22.22 | 36.11 | 41.67 | |
| 2 | 2 | 3 | 36 | 41 |
| | 2.22 | 3.33 | 40.00 | 45.56 |
| | 20.00 | 18.75 | 56.25 | |
| | 4.88 | 7.32 | 87.80 | |
| 3 | 0 | 0 | 13 | 13 |
| | 0.00 | 0.00 | 14.44 | 14.44 |
| | 0.00 | 0.00 | 20.31 | |
| | 0.00 | 0.00 | 100.00 | |
| | 10 | 16 | 64 | 90 |
| | 11.11 | 17.78 | 71.11 | |

### Tests

| N | DF | -LogLike | RSquare (U) |
|---|---|---|---|
| 90 | 4 | 14.453386 | 0.2024 |

| Test | ChiSquare | Prob > ChiSq |
|---|---|---|
| Likelihood | 28.907 | <.0001 |
| Ratio | | |
| Pearson | 26.041 | <.0001 |

### Example 22

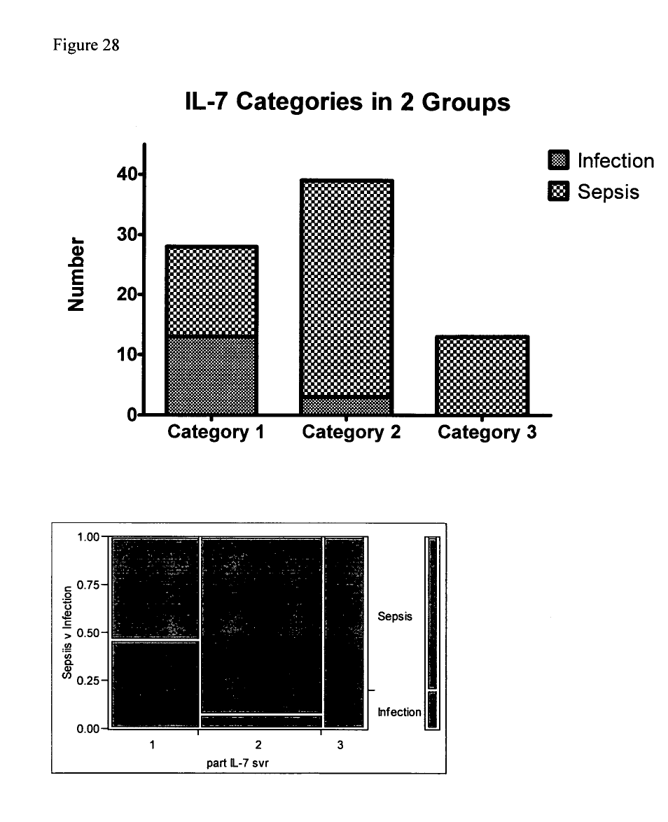

Example 22 gives the distribution of the IL-7 categories, 1 or 2 or 3, in two groups of patients; Patients with Infection, and Patients with Severe sepsis. This example contains a statistical analysis of the distribution of IL-7 categories between the two groups and relates to FIG. 28.

IL-7 levels have been divided into three levels, indicated by the numerals 1, 2 and 3.

The distribution of these IL-2 levels in patients with Infection and Severe sepsis is listed in the table.

| Count | | | |
|---|---|---|---|
| Total % | | | |
| Col % | | | |
| Row % | Infection | Severe sepsis | |
| 1 | 13 | 15 | 28 |
| | 16.25 | 18.75 | 35.00 |
| | 81.25 | 23.44 | |
| | 46.43 | 53.57 | |
| 2 | 3 | 36 | 39 |
| | 3.75 | 45.00 | 48.75 |
| | 18.75 | 56.25 | |
| | 7.69 | 92.31 | |
| 3 | 0 | 13 | 13 |
| | 0.00 | 16.25 | 16.25 |
| | 0.00 | 20.31 | |
| | 0.00 | 100.00 | |
| | 16 | 64 | 80 |
| | 20.00 | 80.00 | |

### Tests

| N | DF | -LogLike | RSquare (U) |
|---|---|---|---|
| 80 | 2 | 10.119177 | 0.2528 |

| Test | ChiSquare | Prob > ChiSq |
|---|---|---|
| Likelihood | 20.238 | <.0001 |
| Ratio | | |
| Pearson | 19.166 | <.0001 |

### Example 23

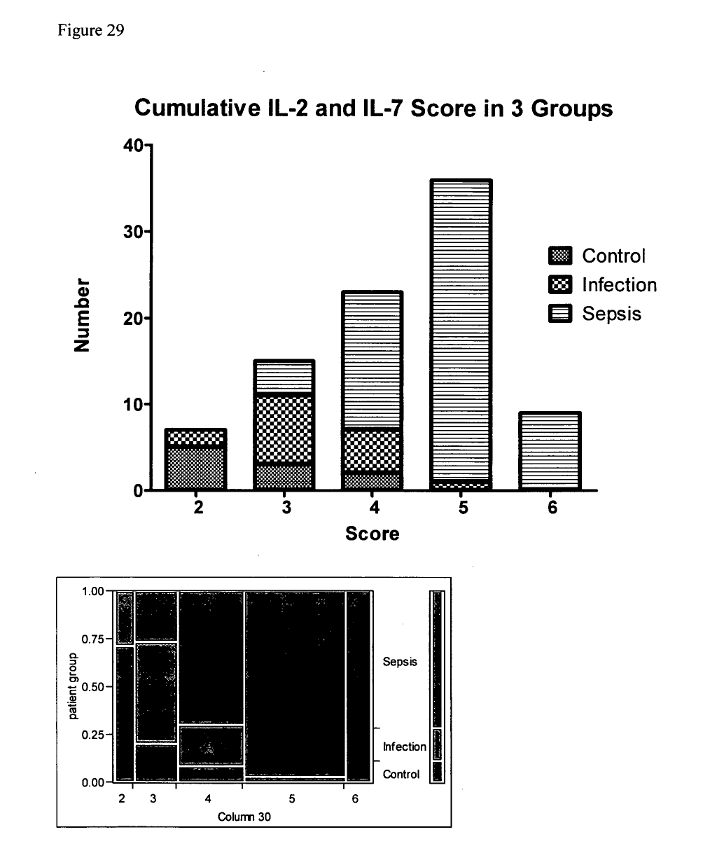

In example 23 the scores for both IL-2 and IL-7 categories have been summated, giving a scoring system which ranges from 2 to 6. The distribution of these scores in the three patient groups, Control, Infection, and Severe sepsis, is statistically analysed and relates to FIG. 29.

In this table the summated score for IL-2 and IL-7 levels is presented as these scores are distributed within 3 groups of patients, Controls, Infection and Severe sepsis

| Count | | | | |
|---|---|---|---|---|
| Total % | | | | |
| Col % | | | | |
| Row % | Control | Infection | Severe sepsis | |
| 2 | 5 | 2 | 0 | 7 |
| | 5.56 | 2.22 | 0.00 | 7.78 |
| | 50.00 | 12.50 | 0.00 | |
| | 71.43 | 28.57 | 0.00 | |
| 3 | 3 | 8 | 4 | 15 |
| | 3.33 | 8.89 | 4.44 | 16.67 |
| | 30.00 | 50.00 | 6.25 | |
| | 20.00 | 53.33 | 26.67 | |
| 4 | 2 | 5 | 16 | 23 |
| | 2.22 | 5.56 | 17.78 | 25.56 |
| | 20.00 | 31.25 | 25.00 | |
| | 8.70 | 21.74 | 69.57 | |
| 5 | 0 | 1 | 35 | 36 |
| | 0.00 | 1.11 | 38.89 | 40.00 |
| | 0.00 | 6.25 | 54.69 | |
| | 0.00 | 2.78 | 97.22 | |
| 6 | 0 | 0 | 9 | 9 |
| | 0.00 | 0.00 | 10.00 | 10.00 |
| | 0.00 | 0.00 | 14.06 | |
| | 0.00 | 0.00 | 100.00 | |
| | 10 | 16 | 64 | 90 |
| | 11.11 | 17.78 | 71.11 | |

### Tests

| N | DF | -LogLike | RSquare (U) |
|---|---|---|---|
| 90 | 8 | 29.204026 | 0.4089 |

| Test | ChiSquare | Prob > ChiSq |
|---|---|---|
| Likelihood | 58.408 | <.0001 |
| Ratio | | |
| Pearson | 60.253 | <.0001 |

### Example 24

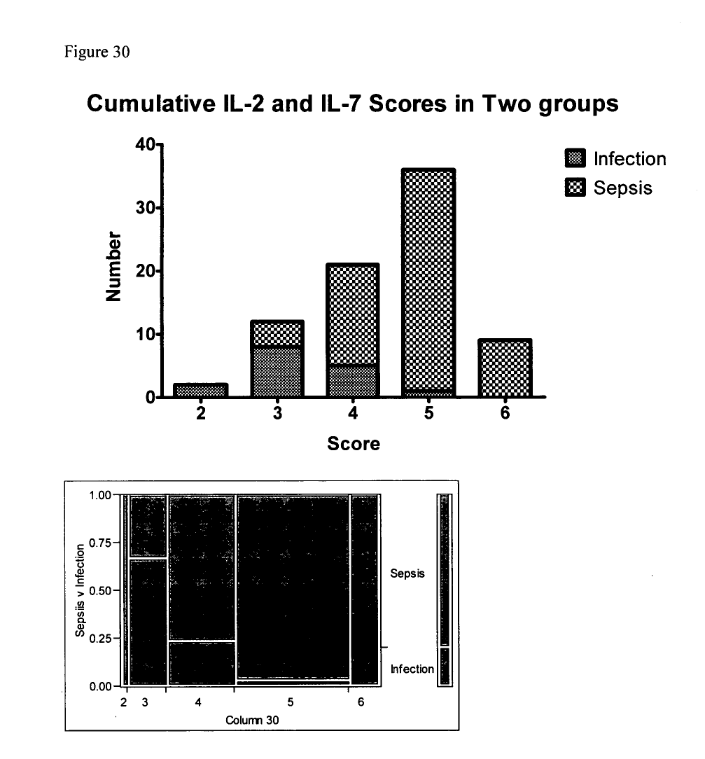

In example 24 the scores for both IL-2 and IL-7 categories have been summated, giving a scoring system which ranges from 2 to 6. The distribution of these scores in the two patient groups, Infection, and Severe sepsis, is statistically analysed. Example 24 relates to FIG. 30.

In this table the summated score for IL-2 and IL-7 levels is presented as these scores are distributed within 2 groups of patients, with Infection and Severe sepsis

Contingency Table
Column 30 By Sepsiis v Infection

| Count | | | |
|---|---|---|---|
| Total % | | | |
| Col % | | | |
| Row % | Infection | Severe sepsis | |
| 2 | 2 | 0 | 2 |
| | 2.50 | 0.00 | 2.50 |
| | 12.50 | 0.00 | |
| | 100.00 | 0.00 | |
| 3 | 8 | 4 | 12 |
| | 10.00 | 5.00 | 15.00 |
| | 50.00 | 6.25 | |
| | 66.67 | 33.33 | |
| 4 | 5 | 16 | 21 |
| | 6.25 | 20.00 | 26.25 |
| | 31.25 | 25.00 | |
| | 23.81 | 76.19 | |
| 5 | 1 | 35 | 36 |
| | 1.25 | 43.75 | 45.00 |
| | 6.25 | 54.69 | |
| | 2.78 | 97.22 | |
| 6 | 0 | 9 | 9 |
| | 0.00 | 11.25 | 11.25 |
| | 0.00 | 14.06 | |
| | 0.00 | 100.00 | |
| | 16 | 64 | 80 |
| | 20.00 | 80.00 | |

### Tests

| N | DF | -LogLike | RSquare (U) |
|---|---|---|---|
| 80 | 4 | 16.298162 | 0.4071 |

| Test | ChiSquare | Prob > ChiSq |
|---|---|---|
| Likelihood | 32.596 | <.0001 |
| Ratio | | |
| Pearson | 33.447 | <.0001 |

The invention is not limited to the embodiments hereinbefore described which may be varied in construction and detail without departing from the spirit of the invention.

1. A method of estimating sepsis risk in an individual with infection comprising a step of assaying a biological sample from the individual for an IL-2 or IL-7 mRNA value, and correlating the mRNA value with sepsis risk.

2. A method as claimed in claim 1, wherein a high IL-2 or IL-7 mRNA value correlates with a low risk of the patient developing sepsis, and wherein a low IL-2 or IL-7 mRNA value correlates with a high risk of the patient developing sepsis.

3. A method as claimed in claim 1 in which the IL-2 or IL-7 mRNA value is quantified by absolute quantification of mRNA copy number, wherein the copy numbers are normalised to a house keeping gene and corrected against a calibration curve for serial dilutions of the IL-2 or IL-7 cDNA.

4. A method as claimed in claim 3 in which the housekeeper gene is selected from β-actin and GAPDH.

5. A method of claim 1 which involves a step of assaying a biological sample from the individual for IL-2 and IL-7 mRNA values, and correlating a sum of the values with sepsis risk.

6. A method as claimed in claim 5 in which the IL-2 mRNA value is the Log 10 of the IL-2 mRNA copy number, and the IL-7 mRNA value is the Log 10 of the IL-7 mRNA copy number, wherein the sum of the mRNA values is correlated with a numerical scale of 3 to 8.5 to provide sepsis risk, wherein 8.5 represents low sepsis risk and 3.5 represents high sepsis risk.

7. A method of claim 1 which involves a step of assaying a biological sample from the individual for a mRNA value of at least two pro-inflammatory cytokines including at least one of IL-2 and IL-7, and at least one of IL-23 and Interferon-γ (INF), and correlating a sum of the mRNA values with sepsis risk.

8. A method as claimed in claim 7 in which the step of correlating the sum of mRNA values with sepsis risk comprises the step of correlating the sum using a logistic regression analysis curve against outcome.

9. A method as claimed in claim 8 in which the mRNA value is a normalised mRNA copy number or a function of the normalised mRNA copy number.

10. A method as claimed in claim 9 in which the function of the normalised mRNA copy number is a Log 10 of the mRNA copy number.

11. A method as claimed in claim 7 in which the at least two pro-inflammatory cytokines are selected from the group consisting of: IL-2 and IL-23; IL-2 and INF; IL-7 and IL-23; IL-7 and INF; IL-2, IL-23, and INF; and IL-7, IL-23, and INF.

12. A method as claimed in claim 10 in which the at least two pro-inflammatory cytokines are IL-2 and IL-23, and wherein the sum of the Log 10 of the mRNA values is correlated with a numerical scale of 5 to 9 to provide sepsis risk, in which 9 represents low sepsis risk and 5 represents high sepsis risk.

13. A method as claimed in claim 10 in which the at least two pro-inflammatory cytokines are IL-2 and INF, and wherein the sum of the Log 10 of the mRNA values is correlated with a numerical scale of 2.5 to 8 to provide sepsis risk, in which 8 represents low sepsis risk and 2.5 represents high sepsis risk.

14. A method as claimed in claim 10 in which the at least two pro-inflammatory cytokines are IL-7 and IL-23, and wherein the sum of the Log 10 of the mRNA values is correlated with a numerical scale of 6 to 10.5 to provide sepsis risk, wherein 10.5 represents low sepsis risk and 6 represents high sepsis risk.

15. A method as claimed in claim 10 in which the at least two pro-inflammatory cytokines are IL-7 and INF, and wherein the sum of the Log 10 of the mRNA values is correlated with a numerical scale of 3.5 to 8.5 to provide sepsis risk, wherein 8.5 represents low sepsis risk and 3.5 represents high sepsis risk.

16. A method as claimed in claim 1 which involves a step of assaying a biological sample from the individual for a mRNA value of at least one pro-inflammatory cytokine including at least one of IL-2 and IL-7, and at least one anti-inflammatory cytokine selected from IL-10 and IL-27, calculating the difference between the pro-inflammatory mRNA value and the anti-inflammatory mRNA value, and correlating the difference with sepsis risk.

17. A method as claimed in claim 16 in which two or more pro-inflammatory cytokines are employed, wherein the mRNA values for the two or more pro-inflammatory cytokines are summated to provide a composite pro-inflammatory mRNA value.

18. A method as claimed in claim 16 in which two or more anti-inflammatory cytokines are employed, wherein the mRNA values for the two or more anti-inflammatory cytokines are summated to provide a composite anti-inflammatory mRNA value.

19. A method as claimed in claim 17 in which the two or more pro-inflammatory cytokines includes at least one of IL-2 or IL-7, and one or more of IL-23 and INF.

20. A method as claimed in claim 16 in which the step of correlating the sum of mRNA values with sepsis risk comprises the step of correlating the sum using a logistic regression analysis curve against outcome.

21. A method as claimed in claim 16 in which the mRNA value is a normalised mRNA copy number or a function of the normalised mRNA copy number.

22. A method as claimed in claim 21 in which the function of the normalised mRNA copy number is a Log 10 of the mRNA copy number.

23. A method as claimed in claim 16 in which the pro-inflammatory and anti-inflammatory combination is selected from the group consisting of: IL-2 and IL-10; IL-2 and IL-27; IL-2, IL-23, INF, and IL-10; IL-2, IL-23, INF, and IL-27; IL-7 and IL-10; IL-7 and IL-27; IL-7, IL-23, INF, and IL-10; IL-7, IL-23, INF, and IL-27.

24. A method as claimed in claim 23 in which the pro-inflammatory cytokine comprises IL-2 and the anti-inflammatory cytokine comprises IL-10, and wherein the difference of the Log 10 of the pro-inflammatory and anti-inflammatory mRNA values is correlated with a numerical scale of −3 to 1.5 to provide sepsis risk, in which 1.5 represents low sepsis risk and −3 represents high sepsis risk.

25. A method as claimed in claim 23 in which the pro-inflammatory cytokine comprises IL-7 and the anti-inflammatory cytokine comprises IL-10, and wherein the difference of the Log 10 of the pro-inflammatory and anti-inflammatory mRNA values is correlated with a numerical scale of −1 to 2.5 to provide sepsis risk, in which 2.5 represents low sepsis risk and −1 represents high sepsis risk.

26. A method as claimed in claim 23 in which the pro-inflammatory cytokines comprise IL-2, IL-23, and INF, and the anti-inflammatory cytokine comprises IL-10, and wherein the difference of the Log 10 of the pro-inflammatory and anti-inflammatory mRNA values is correlated with a numerical scale of 3.5 to 9.5 to provide sepsis risk, in which 9.5 represents low sepsis risk and 3.5 represents high sepsis risk.

27. A method as claimed in claim 23 in which the pro-inflammatory cytokines comprise IL-7, IL-23, and INF, and the anti-inflammatory cytokine comprises IL-10, and wherein the difference of the Log 10 of the pro-inflammatory and anti-inflammatory mRNA values is correlated with a numerical scale of 4.5 to 10.5 to provide sepsis risk, in which 10.5 represents low sepsis risk and 4.5 represents high sepsis risk.

28. A method as claimed in claim 23 in which the pro-inflammatory cytokines comprise IL-7, IL-23, and INF, and the anti-inflammatory cytokines comprise IL-10 and Il-27, and wherein the difference of the Log 10 of the pro-inflammatory and anti-inflammatory mRNA values is correlated with a numerical scale of 1.5 to 7 to provide sepsis risk, in which 7 represents low sepsis risk and 1.5 represents high sepsis risk.

29. A method as claimed in claim 2 in which a high level of IL-2 mRNA expression correlates with IL-2 copy number of at least 570 copy numbers of mRNA.

30. A method as claimed in claim 2 in which a low level of IL-2 expression correlates with an IL-2 copy number of less than 172 copy numbers of mRNA.

31. A method as claimed in claim 2 in which a high level of IL-7 expression correlates with IL-7 copy number of at least 1675 copy numbers of mRNA.

32. A method as claimed in claim 2 in which a low level of IL-2 expression correlates with an IL-7 copy number of less than 283 copy numbers of mRNA.

33. A method as claimed in claim 1 comprising a scoring system in which a patient is assigned a score of 1, 2 or 3 depending on whether the level of expression of IL-2 is high, medium or low, and in which a score of 1 correlates with a IL-2 mRNA copy number of at least 570, a score of 2 correlates with a IL-2 mRNA copy number of from 570 and 172, and a score of 3 correlates with a IL-2 mRNA copy number of less than 172, and wherein a score of 3 correlates with a likelihood of developing severe sepsis, and a score of 1 correlates with a likelihood of not developing severe sepsis.

34. A method as claimed in claim 1 comprising a scoring system in which a patient is assigned a score of 1, 2 or 3 depending on whether the level of expression of IL-7 is high, medium or low, and in which a score of 1 correlates with a IL-7 mRNA copy number of at least 1675, a score of 2 correlates with a IL-7 mRNA copy number of from 1675 and 283, and a score of 3 correlates with a IL-7 mRNA copy number of less than 283, wherein a score of 3 correlates with a likelihood of developing severe sepsis, and a score of 1 correlates with a likelihood of not developing severe sepsis.

35. A method as claimed in claim 33 in which the scoring system involves assaying a patient for IL-2 and IL-7 mRNA levels, assigning a score of 1, 2 or 3 to the patient in respect of each of IL-2 and IL-7, and summating the score to provide a composite score for the patient of between 2 and 6, and wherein a score of 4, 5 or 6 indicates a likelihood that the patient has, or will develop, severe sepsis, and wherein a score of 2 or 3 indicates a likelihood that the patient will not develop severe sepsis.

36. A method as claimed in claim 1 in which the biological sample is a peripheral blood lymphocyte preparation.

37. A method as claimed in claim 36 in which the biological sample is a lymphocyte preparation obtained from lymphocytes contained in the buffy coat layer of a peripheral blood sample.

38. A method of treating or preventing severe sepsis in an individual with infection comprising a step of estimating sepsis risk in an individual with infection using a method of claim 1, and initiating a therapeutic or prophylactic treatment if the sepsis risk is elevated.

39. A method of monitoring a patient undergoing treatment to prevent or ameliorate the development of sepsis, comprising a step of estimating sepsis risk in the individual using a method of claim 1 periodically to monitor effectiveness of the treatment.
